(19) United States
(12) Patent Application Publication
(10) Pub. No.: US 2018/0147274 A1
(43) Pub. Date: May 31, 2018

(54) IPN Virus Genome Mutations and Codon Interactions

(71) Applicant: FVG Limited, Inverness (GB)

(72) Inventor: Oystein Evensen, Oslo (NO)

(21) Appl. No.: 15/561,766

(22) PCT Filed: Mar. 22, 2016
(86) PCT No.: PCT/GB2016/050797

§ 371 Date: Sep. 26, 2017

(30) Foreign Application Priority Data
Mar. 27, 2015 (GB) 1505310.1

(51) Int. Cl. A61K 39/12; C12N 7/00; A61K 39/00

(52) U.S. Cl. CPC A61K 39/12; C12N 7/00; C12N 2720/10034; C12N 2720/10021; A61K 2039/552; C12N 2720/10062; A61K 2039/5254

(57) ABSTRACT

The present invention relates generally to infectious pancreatic necrosis virus (IPNV) and specifically to further characterisation of the genetic stability of IPNV genome. More in particular, the invention relates to greater appreciation and understanding of the frequency of mutagenesis and codon interactions that influence the replication capabilities, virulence and immunogenicity of the virus. There is provided for the first time a method for developing a commercially applicable IPNV vaccine and a vaccine comprising the IPNV genome carrying particular mutations and codons. There are also described uses in prophylaxis or treatment of infectious pancreatic necrosis (IPN) disease.

### FIELD OF THE INVENTION

The present invention relates to further characterisation of the genetic stability of infectious pancreatic necrosis virus (IPNV), in particular to a greater appreciation and understanding of the frequency of mutagenesis and codon interactions that influence the replication capabilities, virulence and immunogenicity of the virus. Thus, a method for developing a commercially applicable IPNV vaccine and a vaccine comprising the IPNV genome carrying the particular mutations and codons for use in prophylaxis or treatment of infectious pancreatic necrosis (IPN) disease are also part of the present invention.

### BACKGROUND OF THE INVENTION

RNA viruses have large genetic diversity and high mutation rates (Steinhauer & Holland 1987). Several biological aspects can be put in the context of these genetic variations, for example virulence, persistence and adaptation both in vitro and in vivo. It is well documented that replication of IPNV relies on a RNA-dependent RNA polymerase which notably lacks the proof reading capabilities of DNA polymerases associated with 3′-5′ exonuclease activity (Duarte et al., 1994; Kohlstaedt et al., 2009; Steinhauser et al., 1992). This natural impediment of the virus results in genetic mutation rates of up to 1 mutation per 10³ bases copied per replication cycle (Drake and Holland, 1999).

In addition and as a further natural deficiency of RNA genome viruses such as IPNV, they have no post-replication error corrections, which gives them low replication fidelity and the possibility of rapid emergence of mutant virus strains (1)—(Domingo, E., C. Escarmis, et al., 1996). These mutations result in a cloud or a population of closely related sequence variants within a host (or several hosts) and can differ by as little as one nucleotide from the average sequence in the population (3)—(Lauring, A. S., and R. Andino 2010). Massive viral infections often results in increased fitness and virulence (1), and movement in the mathematical fitness “landscape”, where new variants will increase to more virulent variants in the virus population (7)—(Wright, S. 1931).

The underlying biological implications of these phenomena have been clearly demonstrated for many viruses like human immunodeficiency virus-1 (HIV), hepatitis C virus (HCV), polio and other virus species. The propensity to mutate is a major challenge for the development of safe live, attenuated RNA virus vaccines.

The IPNV genome consists of two segments of double-stranded RNA that are surrounded by a single-shelled icosahedral capsid of 60 nm in diameter. Genomic segment A (typically 3097 nucleotides) encodes a 106 kDa precursor polyprotein composed of pVP2-VP4-VP3, in that order, and a 15 kDa non-structural VP5 protein, found only in infected cells. Segment B (typically 2777 nucleotides) encodes a minor internal polypeptide VP1 (94 kDa), which is the virion-associated RNA-dependent RNA polymerase (RdRp).

VP2 is a major viral capsid protein and it has been hypothesised that variations in the amino acid residues of this protein may be associated with changes in virulence. In fact, by a comparison of the amino acid sequences of various field isolates exhibiting different mortality in Atlantic salmon fry, the putative motifs involved in virulence of IPNV strains have been proposed. By way of example, such strains typically have residues threonine, alanine, threonine/alanine, and tyrosine/histidine at positions 217, 221, 247 and 500 of the VP2 sequence. Further work has shown that virulent isolates possess residues Thr217 and Ala221; moderate to low virulent strains have Pro217 and Ala221; and strains containing Thr221 are almost always avirulent, irrespective of the residue at position 217.

RNA viruses such as IPNV and IBDV are prone to change through a variety of mechanisms, so it has been assumed that there always is a risk that the virus will revert to greater virulence during multiplication in the vaccinated fish. Such a strategy would require vigilance in monitoring field viruses and the natural history of the disease. So far, unawareness about factors influencing the virulence and genetic stability of IPNV has precluded the use of avirulent strains for vaccine purposes.

Further characterization of the underlying pressures which affect the genetic stability of infectious pancreatic necrosis virus (IPNV) and the frequency of mutagenesis and codon interactions that influence the replication capabilities, virulence and immunogenicity of the virus is much needed for developing suitable avirulent but at the same time immunogenic IPNV vaccines.

The present invention whichhas been described below addresses some of the above identified problems.

### SUMMARY OF THE INVENTION

The present invention provides novel genetic variants of infectious pancreatic necrosis virus (IPNV), in particular variants with greater genome stability which can be used as vaccines in prophylaxis or treatment of infectious pancreatic necrosis (IPN) disease.

It was surpisingly identified by the present inentors that the occurance of certain nucleotides at particular positions in a codon were able to confer a preferred selection of other particular nuclelotides at other particular positions in a codon and thus select for avirulent IPNV which eshibit greater genetic stability. Genetic stability of IPNV is relevant if there are to be selected suitable virla strains for commercial exploitation such that they do not revert to a virulent state. These observations allowed the present inventors to arrive at the present invention. This level of assessment has not been performed prior to this invention.

With the purpose to study the genetic stability of IPNV, different variants made by reverse genetics (clones of virus) were used to establish a persistent infection in Atlantic salmon fry. The strains used represented different virulence categories defined by amino acid position 217 and 221 of the VP2 protein of the virus. A moderately virulent strain carried P₂₁₇A₂₂₁, while an avirulent strain had P₂₁₇T₂₂₁. These strains were used to infect fry and the strains were also combined in the same inoculum (a mixture).

Accordingly, one aspect of the present invention relates to a live avirulent IPNV variant which does not revert to a virulent virus after at least 3 passages or at least 6 passages. According to one embodiment, the live avirulent infectious pancreatic necrosis virus (IPNV) do not revert to a virulent virus after at least 9 passages.

One embodiment relates to a live avirulent IPNV according to the present invention, which when delivered by immersion at a titre of 5×10⁴ TCID₅₀/ml to Atlantic salmon fry held in fresh water at a temperature of 12° C. causes the fry to be virus positive measured by reisolation on RTG-2 cells.

One embodiment relates to a live avirulent IPNV according to the present invention, which when delivered by immersion at a titre of 5×10⁴ TCID₅₀/ml to Atlantic salmon fry held in fresh water at a temperature of 12° C. provides the fry with protection against IPN disease.

One embodiment relates to a live avirulent IPNV according to the present invention, which when delivered by immersion at a titre of 5×10⁴ TCID₅₀/ml to Atlantic salmon fry held in fresh water at a temperature of 12° C. does not cause the fry to develop signs of IPN disease.

In one preferred embodiment, said live avirulent IPNV comprises a nucleic acid encoding a VP2 protein (SEQ ID NO: 2), wherein the amino acid in position 221 of the VP2 protein is Val and not Ala.

In one embodiment, said live avirulent IPNV incorporates a codon at position 220 of the nucleotide sequence coding for VP2 (SEQ ID NO: 3) that is associated with hypervariable codons 217, 221, and 247 of SEQ ID No: 2.

In one further embodiment, said live avirulent IPNV incorporates at position 3 of codon 220 (codon 220.3) is associated with a statistically significant interaction value with that at position 1 of nucleotide codons 247, 217 and position 3 of nucleotide codon 221 (221.3).

In one further embodiment, said avirulent IPNV, the presence of G (guanine) or A (adenosine) at position 3 of codon 220 (codon 220.3) is associated with a statistically significant interaction value with position 1 of nucleotide codons 247 and 217, and position 3 of nucleotide codon 221 (221.3).

In one yet furthe rembodiment, said live avirulent IPNV incorporates a combination of codones selected from T₂₁₇A₂₂₁T₂₄₇.

In one preferred embodiment said codons cause the avirulent INPV not to revert to a virulent virus after at least 3 passages.

One preferred embodiment relates to the live avirulent IPNV according to the present invention, which when delivered by immersion at a titre of 5×10⁴ TCID5o/ml to Atlantic salmon fry held in fresh water at a temperature of 12° C.
causes the fry to be virus positive measured by reisolation on RTG-2 cells;
causes the fry to be virus negative measured by immunohistochemistry;
provides the fry with protection against IPN disease as compared to non-infected fry; and
does not cause the fry to develop any signs of IPN disease.

According to a second aspect the present invention relates to a method of identifying an avirulent IPNV, the method comprising the steps of:
a) characterising the distribution of nucleotides of VP2;
b) correlating step a) with the distribution of nucleotides at codons starting at codon 200 to codon 320; and
c) identifying an avirulent IPNV.

A second aspect the present invention relates to a method of identifying an avirulent IPNV, the method comprising the steps of:
a) characterising the distribution of nucleotides at codons 220;
b) correlating the findings from step a) with the distribution of nucleotides at codons 247, 217 and 221; and
c) identifying an avirulent IPNV.

According to one embodiment of the second aspect, there is provided a method of identifying an avirulent IPNV, the method comprising the steps of:
a) characterising position 3 of codon 220 (220.3);
b) correlating the findings from step a) with position 1 of codons 247 (247.1) and 217 (217.1) and position 3 of codon 221 (221.3) and
c) identifying an avirulent IPNV.

A third aspect of the present invention relates to a live avirulent IPNV according to the present invention or to the IPNV obtained from the method of the present invention, for use as a vaccine.

A fourth aspect of the present invention relates to a live avirulent IPNV according to the present invention, the IPNV obtained from the process of the present invention or a vaccine according to the present invention for use as a vaccine against IPN disease.

A fifth aspect of the present invention relates to a live avirulent IPNV according to the present invention, the IPNV obtained from the method of the present invention or a vaccine according to the present invention for use as a vaccine against IPN disease, wherein distribution is by immersion, oral administration or injection.

Preferred embodiments of the present invention are depicted in the dependent claims and in the detailed description of the invention.

### DESCRIPTION OF THE FIGURES

Preferred embodiments of the present invention will now be illustrated in more detail with reference to the accompanying figures.

FIG. 1. PA/PV/PT challenge group, results from 1^{st} sampling (7d post onset of stress) with codon combinations in the upper window and corresponding amino acids in the lower window. The same procedure was repeated for each examined individual.

FIG. 2A PA (/PV) challenged fish; position 217. Amino acid variability during the first 6 months post challenge. Note that T (threonine) occurs in position 217 by 6 mo post infection; and

FIG. 2B PA (/PV) challenged fish, position 217. Amino acid variability during the stress period. Right column are infected, non-stressed fish (stress control). As can be seen, there is a tendency that stress decreases variability. For the PA/PV-challenged fish position 217 remains with low variability during the first 3 months post challenge, increasing at later stage and particularly beyond 6 months (FIG. 2) of both stressed and non-stressed groups.

FIG. 3A PA (/PV) challenged fish; position 221. Amino acid combinations in residue 221 is dominated by A and V at early time, and with T (threonine) being found at 6 mo pi. Some fluctuation over time; and FIG. 3B. PA (/PV) challenged fish; position 221. Increasing variability beyond 6 mo pi, with less diversity in non-stressed fish (right column), i.e. there is an indication that stress results in some clones get the upper hand. Position 221 was analysed using the same methods and the findings are similar to what is seen for position 217, expect the amino acids differ (FIG. 3). A and V are almost at 1:1 ratio at early time post challenge, with more diversification at later time points.

FIG. 4A. PA(/PV) challenged fish, relative frequency of amino acids in position 217 post challenge. S indicates stress period running for 4 weeks starting at 6 mo pi; and FIG. 4B. PA(/PV) challenged fish, relative frequency of amino acids in position 221 at different time post challenge. S indicates stress period running for 4 weeks starting at 6 mo pi. When results for positions 217 and 221 are combined (FIG. 4), P (217) and V (221) dominate up to 6 months post challenge while there is a shift to T (217) and A (221) combinations beyond 6 months.

FIG. 5A. PA(/PV) challenged fish, relative frequency of amino acids in position 217 post challenge. NS indicates these fish were not stressed (controls), and FIG. 5B. PA(/PV) challenged fish, relative frequency of amino acids in position 221 at different time post challenge. NS indicates these fish were not stressed (controls). When these findings are compared to the PA/PV infected, non-stressed control fish, the same pattern emerges, with a shift from PV dominance at early time post challenge, shifting towards a higher frequency of TA variants beyond 6 months pi (FIG. 5).

FIG. 6A. PT-challenged fish, relative frequency of amino acids in position 217 post challenge (up to 2 months), and FIG. 6B. PT-challenged fish, relative frequency of amino acids in position 217 post challenge, 3-6 months post challenge. There is variation over time and with S in 217 being the dominating amino acid at 6 mo pi. PT challenged fish. The variation in position 217 is less in PT compared to PA/PV challenged fish but still with some variation already at early time post challenge (FIG. 6).

FIG. 7. PT-challenged fish, relative frequency of amino acids in position 217 during the stress period (stressed and non-stressed controls are shown). 3w ps was not available for the non-stressed group.

FIG. 8A, 8B & 8C. PT-challenged fish, relative frequency of amino acids in position 221 post infection and during the stress period (stressed and non-stressed controls are shown). Clearly, T dominates in p221 with an increasing variability with time. In position 221, T dominates at early time post challenge and with increasing variability at later time points (FIG. 8).

### BRIEF DESCRIPTION OF THE SEQUENCES

SEQ ID No: 1 - the nucleotide (RNA) sequence of G700 strain (deposited under ECACC-
No. 11 041201; WO2012/078051 is incorporated by reference in its entirety for all
purposes).
SEQ ID No: 2 - peptide (three letter amino acid abbreviation) sequence of VP2
precursor
Met Asn Thr Asn Lys Ala Thr Ala Thr Tyr Leu Lys Ser Ile Met Leu Pro Glu Thr Gly Pro
Ala Ser Ile Pro Asp Asp Thr Thr Glu Arg His Ile Leu Lys Gln Glu Thr Ser Ser Tyr Asn
Leu Glu Val Ser Glu Ser Gly Ser Gly Ile Leu Val Cys Phe Pro Gly Ala Pro Gly Ser Arg
Val Gly Ala His Tyr Arg Trp Asn Ala Asn Gln Thr Gly Leu Glu Phe Asp Gln Trp Leu
Glu Thr Ser Gln Asp Leu Lys Lys Ala Phe Asn Tyr Gly Arg Leu Ile Ser Arg Lys Tyr Asp
Ile Gln Ser Ser Thr Leu Pro Ala Gly Leu Tyr Ala Leu Asn Gly Thr Leu Asn Ala Ala Thr
Phe Glu Gly Ser Leu Ser Glu Val Glu Ser Leu Ala Tyr Asn Ser Leu Met Ser Leu Thr Thr
Asn Pro Gln Asp Lys Val Asn Asn Gln Leu Val Thr Lys Gly Val Thr Val Leu Asn Leu
Pro Thr Gly Phe Asp Lys Pro Tyr Val ArgLeu Glu Asp Glu Thr Pro Gln Gly Ile Gln Ser
Met Asn Gly Ala Lys Met Arg Cys Thr Ala Ala Ile Ala Pro Arg Arg Tyr Glu Ile Asp Leu
Pro Ser Gln Arg Leu Pro Pro Val Pro Ala Thr Gly Thr Leu Thr Thr Leu Tyr Glu Gly Asn
Ala Asp Ile Val Asn Ser Thr Thr Val Thr Gly Asp Ile Asn Phe Ser Leu Ala Glu Gln Pro
Ala Asn Glu Thr Lys Phe Asp Phe Gln Leu Asp Phe Met Gly Leu Asp Asn Asp Val Pro
Val Val Thr Val Val Ser Ser Val Leu Ala Ser Asp Asp Asn Tyr Arg Gly Val Ser Ala Lys
Met Thr Gln Ser Ile Pro Thr Glu Asn Ile Thr Lys Pro Ile Thr Arg Val Lys Leu Ser Tyr
Lys Ile Asn Gln Gln Thr Glu Ile Gly Asn Val Ala Thr Leu Gly Thr Met Gly Pro Ala Ser
Val Ser Phe Ser Ser Gly Asn Gly Asn Val Pro Gly Val Leu Arg Pro Ile Thr Leu Val Ala
Tyr Glu Lys Met Thr Pro Leu Ser Ile Leu Thr Val Ala Gly Val Ser Asn Tyr Glu Leu Ile
Pro Asn Pro Glu Leu Leu Lys Asn Met Val Thr Arg Tyr Gly Lys Tyr Asp Pro Glu Gly
Leu Asn Tyr Ala Lys Met Ile Leu Ser His Arg Glu Glu Leu Asp Ile Arg Thr Val Trp Arg
Thr Glu Glu Tyr Lys Glu Arg Thr Arg Val Phe Asn Glu Ile Thr Asp Phe Ser Ser Asp Leu
Pro Thr Ser Lys Ala Trp Gly Trp Arg Asp Ile Val Arg Gly Ile Arg Lys Val Ala Ala Pro
Val Leu Ser Thr Leu Phe Pro Met Ala Ala Pro Leu Ile Gly Met Ala Asp Gln Phe Ile Gly
Asp Leu Thr Lys Thr Asn Ala Ala Gly Gly Arg Tyr His Ser Met Ala Ala Gly Gly Arg
Tyr Lys Asp Val Leu Glu Ser Trp Ala
SEQ ID No: 3 - entire A segment - DNA SEQUENCE 3097 BP; 895 A; 928 C;
796 G; 478 T; 0 OTHER - comprising NVI-015PA;
GGAAAGAGAG TTTCAACGTT AGTGGTAACC CACGAGCGGAGAGCTCTTAC
GGAGGAGCTCTCCGTCGATG GCGAAAGCCC TTTCTAACAA ACAAACAAAC
AATCTATATC AATGCAAGAT GAACACAAAC AAGGCAACCG CAACTTACCT
GAAATCCATT ATGCTTCCAG AGACTGGACC AGCAAGCATC CCGGACGACA
TAACGGAGAG ACACATCTTA AAACAAGAGA CCTCGTCATACAACTTAGAG
GTCTCCGAATCAGGAAGTGGCATTCTTGTTTGTTTCCCTGGGGCACCAGGCT
CACGGATCGGTGCACACTACAGATGGAATGCGAACCAGACGGGGCTGGAGT
TCGACCAGTGGCTGGAGACGTCGCAGGACCTGAAGAAAGCCTTCAACTACG
GGAGGCTGATCTCAAGGAAATATGACATCCAAAGCTCCACACTACCGGCCG
GTCTCTATGCTCTGAACGGGACGCTCAACGCTGCCACCTTCGAAGGCAGTCT
GTCTGAGGTGGAGAGCCTGACCTACAACAGCCTGATGTCCCTAACAACGAA
CCCCCAGGACAAAGTCAACAACCAGCTGGTGACCAAAGGAGTCACAGTCCT
GAATCTACCAACAGGGTTCGACAAACCATACGTCCGCCTAGAGGACGAGAC
ACCCCAGGGT CTCCAGTCAA TGAACGGGGC CAAGATGAGGTGCACAGCTG
CAATTGCACCGCGGAGGTACGAGATCGACCTCCCATCCCAACGCCTACCCC
CCGTTACTG CGACAGGAGCCCTCACCACT CTCTACGAGG GAAACGCCGA
CATCGTCAAC TCCACGACAG TGACGGGAGACATAAACTTC AGTCTGACAG
AACAACCCGC AGTCGAGACC AAGTTCGACT TCCAGCTGGACTTCATGGGC
CTTGACAACGACGTCCCAGTTGTCACAGTGGTCAGCTCCGTGCTGGCCACAA
ATGACAAC TACAGAGGAG TCTCAGCCAA GATGACCCAG TCCATCCCGA
CCGAGAACATCACAAAGCCG ATCACCAGGG TCAAGCTGTC ATACAAGATC
AACCAGCAGACGGCAATCGGCAACGTCGCCACCCTGGGCACAATGGGTCCA
GCATCCGTCTCCTTCTCATCAGGGAACGGAAATGTCCCC GGCGTGCTCA
GACCAATCAC ACTGGTGGCC TATGAGAAGA TGACACCGCTGTCCATCCTG
ACCGTAGCTG GAGTGTCCAA CTACGAGCTG ATCCCAAACC
CAGAACTCCTAAAGAACATG GTGACACGCT ATGGCAAGTA CGACCCCGAA
GGTCTCAACT ATGCCAAGATGATCCTGTCC CACAGGGAAG AGCTGGACAT
CAGGACAGTG TGGAGGACAGGGAGTACAAGGAGAGGACC
AGAGTCTTCAACGAAATCACGGACTTCTCCAGTGACCTGCCCACGTCAAAG
GCATGGGGC TGGAGAGACA TAGTCAGAGG AATTCGGAAA GTCGCAGCTC
CTGTACTGTCCACGCTGTTT CCAATGGCAG CACCACTCAT AGGAATGGCA
GACCAATTCA TTGGAGATCTCACCAAGACC AACGCAGCAG GCGGAAGGTA
CCACTCCATG GCCGCAGGAG GGCGCTACAAAGACGTGCTC GAGTCCTGGG
CAAGCGGAGG GCCCGACGGA AAATTCTCCC GAGCCCTCAAGAACAGGCTG
GAGTCCGCCA ACTACGAGGA AGTCGAGCTT CCACCCCCCT CAAAAGGAGT
CATCGTCCCT GTGGTGCACA CAGTCAAGAG TGCACCAGGC GAGGCATTCG
GGTCCCTGGCAATCATAATT CCAGGGGAGT ACCCCGAGCT TCTAGATGCC
AACCAGCAGG TCCTATCCCACTTCGCAAAC GACACCGGGA GCGTGTGGGG
CATAGGAGAG GACATACCCT TCGAGGGAGACAACATGTGC TACACTGCAC
TCCCACTCAA GGAGATCAAA AGAAACGGGAACATAGTAGTCGAGAAGATC
TTTGCTGGACCAATCATGGGTCCCTCTGCTCAACTAGGACTGTCCCTACTAG
TGAACGAC ATCGAGGACG GAGTTCCAAG GATGGTATTC ACCGGCGAAA
TCGCCGATGACGAGGAGACA ATCATACCAA TCTGCGGTGT AGACATCAAA
GCCATCGCAG CCCATGAACAAGGGCTGCCA CTCATCGGCA ACCAACCAGG
AGTGGACGAG GAGGTGCGAA ACACATCCCTGGCCGCACAC CTGATCCAGA
CCGGAACCCT GCCCGTACAA CGCGCAAAGGGCTCCAACAAGAGGATCAAG
TACCTGGGAGAGCTGATGGCATCAAATGCATCCGGGATGGACGAGGAACTG
CAACGCCTC CTGAACGCCA CAATGGCACG GGCCAAAGAA GTCCAGGACG
CCGAGATCTACAAACTTCTT AAGCTCATGG CATGGACCAG AAAGAACGAC
CTCACCGACC ACATGTACGAGTGGTCAAAA GAGGACCCCG ATGCACTAAA
GTTCGGAAAG CTCATCAGCA CGCCACCAAAGCGCCCCGAG AAGCCCAAAG
GACCAGACCA ACACCATGCC CAAGAGGCGA GAGCCACCCGCATATCACTG
GACGCCGTGAGAGCCGGGGCGGACTTCGCCACACCGGAATGGGTCGCGCTG
AACAACTAC CGCGGCCCAT CTCCCGGGCA GTTCAAGTAC TACCTGATCA
CTGGACGAGAACCAGAACCA GGCGACGAGTACGAGGACTACATAAAACAA
CCCATTGTGA AACCGACCGACATGAACAAA ATCAGACGTC TAGCCAACAG
TGTGTACGGC CTCCCACACC AGGAACCAGCACCAGAGGAG TTCTACGATG
CAGTTGCAGC TGTATTCGCA CAGAACGGAG GCAGAGGTCCCGACCAGGAC
CAAATGCAAGACCTCAGGGAGCTCGCAAGACAGATGAAACGACGACCCCG
GAACGCCGAT GCACCACGGAGAACCAGAGCGCCAGCGGAACCGGCACCGC
CCGGACGCTCAAGGTTCACC CCCAGCGGAG ACAACGCTGA GGTGTAACGA
CTACTCTCTT TCCTGACTGATCCCCTGGCC AAAACCCCGG CCCCCCAGGG
GGCCCCC
SEQ ID No: 4 - peptide (single letter amino acid
abbreviation) sequence of NVI-015 (TAT-VTNA)
MNTNKATATY LKSIMLPETG PASIPDDITE RHILKQETSS YNLEVSESGS
GILVCFPGAPGSRIGAHYRW NANQTGLEFD QWLETSQDLK KAFNYGRLIS
RKYDIQSSTL PAGLYALNGTLNAATFEGSL SEVESLTYNS LMSLTTNPQD
KVNNQLVTKG VTVLNLPTGF DKPYVRLEDETPQGLQSMNG AKMRCTAAIA
PRRYEIDLPS QRLPPVTATG ALTTLYEGNA DIVNSTTVTGDINFSLTEQP
AVETKFDFQLDFMGLDNDVPVVTVVSSVLATNDNYRGVSAKMTQSIPTENITK
PITRVKLSYKINQQTAIGNVATLGTMGPASVSFSSGNGNVPGVLRPITLVAYEK
MTPLSILTVAGVSNYELIPNPEL LKNMVTRYGK YDPEGLNYAK MILSHREELD
IRTVWRTEEYKERTRVFNEI TDFSSDLPTS KAWGWRDIVR GIRKVAAPVL
STLFPMAAPL IGMADQFIGDLTKTNAAGGR YHSMAAGGRY KDVLESWA
SEQ ID No: 5 - peptide (asingle letter mino acid
abbreviation) sequence og NVI-025 which is a PAA-VTNA
variant. SEQUENCE 508 AA; 55616 MW; 1372267 CN
(TRANSLATED FROM DNA SEQUENCE 25A001 (BASES 97 TO 3012).
SQ SEQUENCE 508 AA; 55616 MW; 1372267 CN):
MNTNKATATY LKSIMLPETG PASIPDDITE RHILKQETSS YNLEVSESGS
GILVCFPGAPGSRIGAHYRW NANQTGLEFD QWLETSQDLK KAFNYGRLIS
RKYDIQSSTL PAGLYALNGTLNAATFEGSL SEVESLTYNS LMSLTTNPQD
KVNNQLVTKG VTVLNLPTGF DKPYVRLEDETPQGLQSMNG AKMRCTAAIA
PRRYEIDLPS QRLPPVPATG ALTTLYEGNA DIVNSTTVTGDINFSLAEQP
AVETKFDFQL DFMGLDNDVP VVTVVSSVLA TNDNYRGVSA KMTQSIPTEN
ITKPITRVKL SYKINQQTAI GNVATLGTMG PASVSFSSGN GNVPGVLRPI
TLVAYEKMTPLSILTVAGVS NYELIPNPEL LKNMVTRYGK YDPEGLNYAK
MILSHREELD IRTVWRTEEYKERTRVFNEI TDFSSDLPTS KAWGWRDIVR
GIRKVAAPVL STLFPMAAPL IGMADQFIGD LTKTNAAGGR YHSMAAGGRH
KDVLESWA
SEQ ID No: 6 - Forward IPNV1F primer -
ATCTGCGGAGTAGACATCAAAG
SEQ ID No: 7 Reverse primer - IPNV2R -
TGCAGTTCTTCGTCCATCCC
SEQ ID No: 8 - A-Sp500 forward primer -
GAGTCACAGTCCTGAATC
SEQ ID No: 9 DNA - entire A segment - comprising NVI-025.
GGAAAGAGAG TTTCAACGTT AGTGGCAACC CACGAGCGGA
GAGCTCCTAC GGAGGAGCTCTCCGTCGATG GCGAAAGCCC TTTCTAACAA
ACAAACAAAC AATCTATATC AATGCAAGATGAACACAAAC
AAGGCAACCG CAACTTACCT GAAATCCATT ATGCTTCCAG
AGACTGGACCAGCAAGCATC CCGGACGACA TAACGGAGAG ACACATCTTA
AAACAAGAGA CCTCGTCATACAACTTAGAG GTCTCCGAAT CAGGAAGTGG
CATTCTTGTT TGTTTCCCTG GGGCACCAGGCTCACGGATC GGTGCACACT
ACAGATGGAA TGCGAACCAG ACGGGGCTGG
AGTTCGACCAGTGGCTGGAG ACGTCGCAGG ACCTGAAGAA AGCCTTCAAC
TACGGGAGGT TGATCTCAAGGAAATACGAC ATCCAAAGCT CCACACTACC
GGCCGGTCTC TATGCTCTGA ACGGGACGCTCAACGCTGCC ACCTTCGAGG
GCAGTCTGTC TGAGGTGGAG AGCCTGACCT ACAACAGCCTGATGTCCCTA
ACAACGAACC CCCAGGACAA AGTCAACAAC CAGCTGGTGA
CCAAAGGAGTCACAGTCCTG AATCTACCAA CAGGGTTCGA CAAGCCATAC
GTCCGCCTAG AGGACGAGACACCCCAGGGT CTCCAGTCAA TGAACGGGGC
CAAGATGAGG TGCACAGCTG CAATTGCACCGCGGAGGTAC GAGATCGACC
TCCCATCCCA ACGCCTACCC CCCGTTCCTG CGACAGGGGCCCTCACCACT
CTCTACGAGG GAAACGCCGA CATCGTCAAC TCCACGACAG
TGACGGGAGACATAAACTTC AGTCTGGCAG AACAACCCGC AGTCGAGACC
AAGTTCGACT TCCAGCTGGACTTCATGGGC CTTGACAACG ACGTCCCAGT
CGTCACAGTG GTCAGCTCCG TGCTGGCCACAAATGACAAC TACAGAGGAG
TCTCAGCCAA GATGACCCAG TCCATCCCGA CCGAGAACATCACAAAGCCG
ATCACCAGGG TCAAGCTGTC ATACAAGATC AACCAGCAGA
CAGCAATCGGCAACGTCGCC ACCCTGGGCA CAATGGGTCC AGCATCCGTC
TCCTTCTCAT CAGGGAACGGAAATGTCCCC GGCGTGCTCA GACCAATCAC
ACTGGTGGCC TATGAGAAGA TGACACCGCTGTCCATCCTG ACCGTAGCTG
GAGTGTCCAA CTACGAGCTG ATCCCAAACC CAGAACTCCTCAAGAACATG
GTGACACGCT ATGGCAAGTA CGACCCCGAA GGTCTCAACT
ATGCCAAGATGATCCTGTCC CACAGGGAAG AGCTGGACAT CAGGACAGTG
TGGAGGACAG AGGAGTACAAGGAGAGGACC AGAGTCTTCA
ACGAAATCAC GGACTTCTCC AGTGACCTGC CCACGTCAAAGGCATGGGGC
TGGAGAGACA TAGTCAGAGG AATTCGGAAA GTCGCAGCTC
CTGTACTGTCCACGCTGTTT CCAATGGCAG CACCACTCAT
AGGAATGGCAGACCAATTCA TTGGAGATCTCACCAAGACC AACGCAGCAG
GCGGAAGGTA CCACTCCATG GCCGCAGGAG GGCGCCACAAAGACGTGCTC
GAGTCCTGGG CAAGCGGAGG GCCCGACGGA AAATTCTCCC
GAGCCCTCAAGAACAGGCTG GAGTCCGCCA ACTACGAGGA AGTCGAGCTT
CCACCCCCCT CAAAAGGAGTCATCGTCCCT GTGGTGCACA CAGTCAAGAG
TGCACCAGGC GAGGCATTCG GGTCCCTGGCAATCATAATT CCAGGGGAGT
ACCCCGAGCT TCTAGATGCC AACCAGCAGG TCCTATCCCACTTCGCAAAC
GACACCGGGA GCGTGTGGGG CATAGGAGAG GACATACCCT
TCGAGGGAGACAACATGTGC TACACTGCAC TCCCACTCAA GGAGATCAAA
AGAAACGGGA ACATAGTAGTCGAGAAGATC TTTGCTGGAC CAATCATGGG
TCCCTCTGCT CAACTAGGAC TGTCCCTACTTGTGAACGAC ATCGAGGACG
GAGTTCCAAG GATGGTATTC ACCGGCGAAA TCGCCGATGACGAGGAGACA
ATCATACCAA TCTGCGGTGT AGACATCAAA GCCATCGCAG
CCCATGAACAAGGGCTGCCA CTCATCGGCA ACCAACCAGG AGTGGACGAG
GAGGTGCGAA ACACATCCCTGGCCGCACAC CTGATCCAGA CCGGAACCCT
GCCCGTACAA CGCGCAAAGGGCTCCAACAAGAGGATCAAG TACCTGGGAG
AGCTGATGGC ATCAAATGCA TCCGGGATGG ACGAGGAACTGCAACGCCTC
CTGAACGCCA CAATGGCACG GGCCAAAGAA GTCCAGGACG
CCGAGATCTACAAACTTCTT AAGCTCATGG CATGGACCAG AAAGAACGAC
CTCACCGACC ACATGTACGAGTGGTCAAAA GAGGACCCCG ATGCACTAAA
GTTCGGAAAG CTCATCAGCA CGCCACCAAAGCACCCTGAG AAGCCCAAAG
GACCAGACCA ACACCACGCC CAAGAGGCGA GAGCCACCCGCATATCACTG
GACGCCGTGA GAGCCGGGGC GGACTTCGCC ACACCGGAAT
GGGTCGCGCTGAACAACTAC CGCGGCCCAT CTCCCGGGCA GTTCAAGTAC
TACCTGATCA CTGGACGAGAACCAGAACCA GGCGACGAGT
ACGAGGACTA CATAAAACAA CCCATTGTGA
AACCAACCGACATGAACAAA ATCAGACGTC TAGCCAACAG TGTGTACGGC
CTCCCACACC AGGAACCAGCACCAGAGGAG TTCTACGATG CAGTTGCAGC
TGTATTCGCA CAGAACGGAG GCAGAGGTCCCGACCAGGAC
CAAATGCAAG ACCTCAGGGA GCTCGCAAGA CAGATGAAAC
GACGACCCCGGAACGCCGAT GCACCACGGA GAACCAGAGC
GCCAGCGGAA CCGGCACCGC CCGGACGCTCAAGGTTCACC CCCAGCGGAG
ACAACGCTGA GGTGTAACGA CTACTCTCTT TCCTGACTGATCCCCTGGCC
AAAACCCCGG CCCCCCAGGG GGCCCCC

### DETAILED DESCRIPTION OF THE INVENTION

An ideal vaccine for IPNV should induce long lasting protection at an early age, prevent virulent carrier formation and be effective against a large number of IPNV serotypes. Injection cannot be used for small fish, therefore either oral delivery or immersion are most preferred routes for early vaccination. In addition, it must be genetically stable such that it does not revert to a virulent viral strain before it can facilitate the development of an immune response to subsequent IPNV exposure.

Previously it has been assumed that these attributes of an ideal IPNV vaccine must be met either by a recombinant subunit vaccine or by an inactivated viral vaccine, as a live avirulent vaccine could potentially lead to virulent carrier formation and disease in case the avirulent virus should revert to a virulent virus. In this regard, the present inventors have alse reported the development of an avirulent IPNV strain which induces long lasting protection at an early age, prevents virulent carrier formation, is effective against a large number of IPNV serotypes and may be delivered by, injection, oral administration or by immersion, preferably the latter (G700 strain which was deposited under ECACC-No. 11 041201; WO2012/078051 is incorporated by reference in its entirety for all purposes). Interestingly, the fish infected with the G700 strain seemed to be protected from developing IPN when mixed with fish carrying the virulent strain. These findings revealed a potential for this field strain as a live avirulent vaccine against IPN.

In order to further characteris the underlying genetic mechanisms which influence the genetic stability of IPNV avirulent strains, the present inventors chose to stury this in greater detail by using a combination of statistical and in vivo correlation experiments to try and understand whether there are particular regions within the viral genome e.g. within the VP2 protein, which would affect or otherwise contribute to the preferential selection of a particular genotype over another.

In aprticular they found that some codones within the VP2 gene if already selected would influence other sequences such that a more avirulent or less virulent IPNV strain is eventualy propagated using their methods.

In the context of the invention, the term “reconstructed” or “recombinant” in relation to a sequence, nucleic acid, indicates a sequence, nucleic acid or unit which does not exist naturally in the virus and has been assembled and/or inserted in said virus or an ancestor thereof, using recombinant DNA technology, (also called gene cloning or molecular cloning) which refers to techniques of transfer of DNA from one organism to another.

The term “recombinant” in relation to a IPNV refers to an existing IPNV whose genome has been modified by insertion of at least one heterologous nucleic acid sequence, i.e., DNA which corresponds to a gene or part thereof not identical to the nucleic acid sequence of a gene naturally present in the existing virus.

Alternatively, the existing IPNV may be genetically modified by the incorporation into the virus genome of a homologous nucleic acid sequence, i.e., DNA which corresponds to a gene or part thereof identical of a gene naturally present in existing IPN virus. It will be understood that the resulting recombinant IPN virus can be manufactured by a variety of methods, and once made, can be reproduced without use of further recombinant DNA technology.

In the present description, the terms “nucleic acid” “nucleic sequence” and “nucleotide sequence” are used interchangeably and refer to a sequence of deoxyribonucleotides and/or ribonucleotides. The nucleotide sequence may be first prepared by e.g., recombinant, enzymatic and/or chemical techniques, and subsequently replicated in a host cell or an in vitro system. The nucleotide sequence preferentially comprises an open reading frame encoding a peptide. The nucleotide sequence may contain additional sequences such as a transcription terminator, a signal peptide, an IRES, an intron, etc. Preferably, the gene does not contain an intron.

The term “heterologous nucleic acid sequence” refers to a DNA which corresponds to a gene or part thereof not identical to the nucleic acid sequence of a gene naturally present in IPN virus particularly to G700 strain(deposited under ECACC-No. 11 041201; WO02012/078051 is incorporated by reference in its entirety for all purposes). The term “homologous nucleic acid sequence” refers to a DNA which corresponds to a gene or part thereof identical of a gene naturally present in IPNV.

The term “vaccine” as used herein includes an agent which may be used to cause, stimulate or amplify the immune system.

In a first aspect of the present invention relates to a live avirulent infectious pancreatic necrosis virus (IPNV) which preferably do not revert to a virulent virus after at least 3 passages or after at least 6 passages in hosts known to be susceptible to IPNV. According to one embodiment, the live avirulent infectious pancreatic necrosis virus (IPNV) do not revert to a virulent virus after at least 9 passages.

As used herein, the term “live” as applied to viruses refers to a virus that retains the ability of infecting an appropriate host (as opposed to inactivated or subunit vaccines).

As used herein, the term “avirulent” as applied to viruses is understood to mean a virus strain which has substantially lost, preferably completely lost, its ability to cause disease in fish infected with the strain, although its ability to invade fish, i.e. to penetrate into the fish by the usual route of the virus and to reproduce in the body of the fish, remains substantially intact. It is also envisaged that the immunogenisity of the avirulent viruses would be at least maintained or imporved relative to a wilt type virus.

The term “infectious” as applied to viruses indicates that the virus has the ability to reproduce. The virus can be pathogenic or nonpathogenic and still be infectious.

The term “pancreatic necrosis virus (IPNV)” refers to the causative agent of IPN and is a member of the Genus Aquabirnavirus, family Birnaviridae.

The term “virulent” as applied to viruses herein indicates that the virus is pathogenic, meaning that the virus causes disease to its host.

The term “revert to a virulent virus” as applied to avirulent viruses refers to the process where an avirulent virus revert to a pathogenic virus (meaning that the virus cause disease to its host) by naturally occurring processes (usually a mutation in position 217 and/or 221 and/or 220 and/or 247 of the VP2 protein).

Preferably said virus does not revert to a virulent virus after 6, 8, 10, 15, 20, 25, 30, 35 or 50 passages in hosts known to be susceptible to IPNV. More preferably said virus does not revert to a virulent virus after at least 7, 9, 11, 15, 20, 25, 30, 35 or 50 passages in hosts known to be susceptible to IPNV. Most preferably said virus does not revert to a virulent virus.

In one embodiment the VP2 protein is NVI-15PA which is characterised by the amino acid sequence according to SEQ ID No: 4. In another embodiment NVI-15PA is encoded by the nucleotide sequenc of SEQ ID No: 3 or a fragment thereof.

In one further embodiment the VP2 protein is NVI-025 which is charcaterised by the amino acid sequence according to SEQ ID No: 5. In another embodiment NVI-025 is encoded by the nucleotide sequence of SEQ ID No: 9 or a fragment thereof.

In one embodiment according to the first aspect of the present invention, the host is Atlantic salmon (Salmo salar L.) fry preferably of the AquaGen breed.

If the live avirulent IPNV according to the first aspect of the present invention is delivered by immersion at a titre of 5×10⁴ TCID₅₀/ml to Atlantic salmon fry held in fresh water at a temperature of 12° C. it causes the fry to be virus positive measured by reisolation on RTG-2 cells. However, the salmon fry were identified to be virus negative when measured by immunohistochemistry which suggests that the virus is present in the fry only in subclinical levels.

The term “TCID₅₀” as used herein refers to the amount of virus required to produce a cytopathic effect in 50% of inoculated tissue culture cells. Virus infection in cells is complex and results in many changes to the host cell, known collectively as the cytopathic effect (CPE). Such changes include altered shape, detachment from substrate, lysis, membrane fusion, altered membrane permeability, inclusion bodies and apoptosis. The method used to determine the TCID₅₀ value is well known to a man skilled in the art (Beitrag zur kollektiven Behandlung pharmakologischer Reihenversueche, Kärber G., vol. 162, 1931).

Even though the virus was present in the salmon fry only in subclinical levels, the live avirulent IPNV according to the first aspect of the present invention has been shown to provide the fry with protection against IPN disease, in particular as compared to fry which have not been exposed to the live avirulent IPNV according to the first aspect of the present invention.

Further, if the live avirulent IPNV according to the first aspect of the present invention is delivered by immersion at a titre of 2×10⁵ TCID₅₀/ml to Atlantic salmon fry held in fresh water at a temperature of 12° C. it does not cause the fry to develop any signs of IPN disease, in particular no pathological lesions have been observed in internal organs including the pancreas of the fry (histologically).

As previously disclosed, IPNV has typically two structural proteins which form the IPNV capsid. Virus protein 2 (VP2) of IPNV is one out of these two structural proteins and has been shown to be responsible for the production of type-specific monoclonal antibodies. It has previously been suggested that the sequence of the VP2 protein decides whether the virus is virulent or avirulent, and in case of the latter it has now been shown that certain amino acids of the VP2 protein are important determinants as to whether the avirulent virus may revert to a virulent virus.

In one embodiment according to the first aspect of the present invention, said virus comprises a nucleic acid encoding a VP2 protein, wherein the amino acid in position 221 of the VP2 protein is not Ala.

In another embodiment according to the first aspect of the present invention, said virus comprises a nucleic acid encoding a VP2 protein, wherein position 3 of codon 220 is associated with a statistically significant interaction value with position 1 of nucleotide codons 247 (247.1), 217 (217.1) and position 3 of nucleotide codon 221 (221.3).

According to a further embodiment of the first aspect, said virus comprises a nucleic acid encoding a VP2 protein, wherein the presence of G (guanine) or A (adenosine) at position 3 of codon 220 (codon 220.3) is associated with a statistically significant interaction value with position 1 of nucleotide codons 247 (247.1), 217 (217.1), and position 3 of nucleotide codon 221 (221.3).

The term “statistically significant” as used in the context of the present inventino is intended to mean the likelihood that a result or relationship is caused by something other than mere random chance. Statistical testing assessments are preferably employed to determine if a result is statistically significant or not. Different staristical tests would be know to the skilled person.

According to a yet further embodiment of the first aspect, said virus comprises a nucleic acid encoding a VP2 protein, wherein the live avirulent IPNV incorporates a combination of codones selected from T₂₁₇A₂₂₁T₂₄₇. Sequence homologues of IPNV which exhibit avirulent characteristics as contemplated by the present invention are also encompassed within the scope of the present invention.

In one preferred embodiment, said homologues exhibit sequence identity is selected from the group consisting of at least 85% sequence identity, at least 90% sequence identity, at least 95% sequence identity and 100% sequence identity.

The term “sequence identity” indicates a quantitative measure of the degree of homology between two amino acid sequences of equal length or between two nucleotide sequences of equal length. The two sequences to be compared must be aligned to best possible fit possible with the insertion of gaps or alternatively, truncation at the ends of the protein sequences. The sequence identity can be calculated as:

$$\frac{( {N}_{ref} - {N}_{dif} ) 100}{{N}_{ref}}$$

wherein N_{dif} is the total number of non-identical residues in the two sequences when aligned and wherein N_{ref} is the number of residues in one of the sequences. A gap is counted as non-identity of the specific residue(s). Sequence identity can alternatively be calculated by the BLAST program e.g. the BLASTP program (Pearson and Lipman 1988) (www.ncbi.nlm.nih.gov/cgi-bin/BLAST).

In one embodiment of the invention, alignment is performed with the sequence alignment method ClustalW with default parameters as described by Thompson J., et al 1994, available at http://www2.ebi.ac.uk/clustalw/.

According to another aspect, the present invention provides a method for identifying an avirulent IPNV, the method comprising the steps of:
a) characterising the distribution of nucleotides of VP2;
b) correlating step a) with the distribution of nucleotides at codons starting at codon 200 to codon 320; and
c) identifying an avirulent IPNV.

According to one embodiment of the second aspect, there is provided a method for identifying an avirulent IPNV, the method comprising the steps of:
a) characterising the distribution of nucleotides of codons 220;
b) correlating step a) with the distribution of nucleotides at codons 247, 217 and 221; and
c) identifying an avirulent IPNV.

According to a further embodiment of the second aspect there is provided a method for identifying an avirulent IPNV, where the method further comprising the steps of:
a) characterising position 3 of codon 220 (220.3);
b) correlating the findings from step a) with position 1 of codons 247 (247.1) and 217 (217.1) and position 3 of codon 221 (221.3) and
c) identifying an avirulent IPNV.

As used in the context of the present invention the term “correlating” or “correlation” is intended to mean a statistical measure that indicates the extent to which two or more variables, such as the occurrence of a nucleotide in a given position of a codons, which fluctuate together. A positive correlation indicates the extent to which those variables increase or decrease in parallel; a negative correlation indicates the extent to which one variable increases as the other decreases.

Preferably the method of the second aspect identified an avirulent IPNV which does not revert to a virulent virus after 6, 8, 10, 15, 20, 25, 30, 35 or 50 passages in hosts known to be susceptible to IPNV. More preferably said virus does not revert to a virulent virus after at least 7, 9, 11, 15, 20, 25, 30, 35 or 50 passages in hosts known to be susceptible to IPNV. Most preferably said virus does not revert to a virulent virus.

According to another aspect of the present invention there is provided a vaccine, comprising the live avirulent IPNV according to the first aspect of the present invention or an avirulent IPNV obtained by the method of the present invention.

According to another aspect of the present invention there is provided a live avirulent IPNV according to the first aspect of the present invention or an avirulent IPNV obtained by the method of the present invention, for use as a vaccine.

In one preferred embodiment according to this aspect of the present invention, the live avirulent IPNV according to the first aspect of the present invention or an avirulent IPNV obtained by the method of the present invention, is for use as a vaccine against IPN disease in fry and/or smolt and/or fish; more preferably for use as a vaccine against IPN disease in fry.

In one particularily preferred embodiment according to this aspect of the present invention, the live avirulent IPNV according to the first aspect of the present invention or an avirulent IPNV obtained by the method of the present invention is for vaccination of fry against IPN disease.

In one preferred embodiment according to this aspect of the present invention, the vaccine is distributed by immersion, oral administration or injection; more preferably distributed to fry by immersion, oral administration or injection.

In another preferred embodiment according to this aspect of the present invention, said fry are kept in an environment which is free of virulent IPNV for at least 5, 10 or 15 days post vaccination with said avirulent IPNV.

In another preferred embodiment according to this aspect of the present invention, said virus or vaccine is distributed no later than day 4 after start feeding the fry.

In another preferred embodiment according to the third aspect of the present invention, distribution is by immersion using a virus dosage in the range 1×10⁴ TCID₅₀/ml to 1×10⁶ TCID₅₀/ml; more preferably 5×10⁴ TCID₅₀/ml to 5×10⁵ TCID₅₀/ml; even more preferably 1×10⁵ TCID₅₀/ml to 5×10⁵ TCID₅₀/ml; and most preferably about 2×10⁵ TCID₅₀/ml.

The present invention also relates to a live avirulent IPNV for the prophylaxis or treatment of IPN disease.

In a further aspect, the present invention is directed to a method of generating an avirulent IPNV comprising the steps of:
a) characterising the distribution of nucleotides of VP2;
b) correlating step a) with the distribution of nucleotides at codons starting at codon 200 to codon 320; and
c) producing an avirulent IPNV.

The steps of characterising the distribution of nucleotides of the VP2 protein can involve both statistical as well in vitro and/or in vivo experimental verification and testing prosedures. These prosedures have been described in the present invention. It is also contemplated that in silico methodologies may be used in generating and characterising different avirulent strains of IPNV according to the present invention. The different in vitro, in vivo and in silico would be well know to those of skill in the art.

Having now fully described the present invention in some detail by way of illustration and example for purpose of clarity of understanding, it will be obvious to one of ordinary skill in the art that same can be performed by modifying or changing the invention by with a wide and equivalent range of conditions, formulations and other parameters thereof, and that such modifications or changes are intended to be encompassed within the scope of the appended claims.

### Materials and Methods & Results

The following materials and methods & results are inclided as illustrative examples on how to make and use the invention. It is not intended that these examples should limit the scope of the invention in any manner or to any degree. By way of example only the techniques described in the following publication: Molecular cloning: A laboratory manual: Edited by Sambrook, Fritsch and Maniatis—Fourth Edition: 2012 (9), may be relied upon by those aiming to carry out the claimed scope of the present invention.

Cells and Viruses

Chinock salmon embryo (cells (CHSE-214; ATCC CCL-1681) were maintained at 20° C. in L-15 medium (Sigma-Aldrich) supplemented with 5% fetal bovine serum (FBS, Medprobe), 2 mM L-glutamin (Sigma-Aldrich) and 50 □g ml⁻¹ gentamicin (Sigma-Aldrich). Rainbow trout gonad cells (RTG-2; ATCC CCL-55) were grown at 20° C. in L-15 medium supplemented with 10% fetal bovine serum, 2 mM L-glutamin and 50 □g ml⁻¹ gentamicin.

All virus isolates were propagated in RTG-2 cells by inoculation of 100 μl virus stock solution (stored at −20° C. in 30% glycerol, titer 10⁷ TCID₅₀/ml) onto 70% confluent T-162 cm² cell culture flasks. The supernatants were harvested when widespread cytopathic effect was visible, 5-7 days post infection. The cell culture supernatants were obtained after a centrifugation at 2500G for 10 minutes, and sterile filtration (0.22 μl). The infectious titer was determined by end point dilution on CHSE-214 cells, and the TCID₅₀ was estimated by the method of Karber (Karber 1931).

Construction of Full-Length cDNA Clones.

Generation of full-length cDNA clones of the entire coding and non-coding regions of the different isolates of segments A and B was performed according to procedures described previously (4, 6, 8). The genetically engineered virus strains were propagated on RTG-2 monolayers. The supernatants were harvested following centrifugation at 2500G for 10 minutes and sterile filtration (0.22 μl). RNA extraction using the QIAamp viral mini kit (Qiagen) was carried out according to the manufacturer's recommendation. To ensure that the generated clones had the correct residues, the genomes of segment A and B were sequenced (8) and the chromatograms were analyzed as described (2).

Plaque Purification Assay

To generate a PT variant we took advantage of previously observed attenuation characteristics of IPNV seen in CHSE-214 following passage (6). rNVI-15PA was passaged ten times on CHSE-214 cells. The obtained isolate was then plaque purified by inoculating RTG-2 monolayers on six well plates with 10-fold dilution (10⁻³ to 10⁻⁸) of cell culture supernatants. After 1 hr adsorption at room temperature the inoculum was removed and the cells were overlaid with 0.8% SeaPlaque Agarose (BioWhittaker) in 2×L-15 medium (Sigma) containing 5% FBS and 1% L-glutamine. The cells were incubated at 15° C. for 4 days and plaques formed by cytopathic effect (CPE) were picked by insertion of a 3 mm punch biopsy (Miltex) through the agar to the plate.

The plaques were subsequently inoculated on RTG-2 monolayers and incubated at 15° C. until full CPE was observed. Then the supernatant was harvested following a centrifugation at 2500G for 10 minutes and sterile filtration (0.22 μl). RNA was extracted using the QIAamp Viral RNA mini kit according to the manufacturer's recommendations. Complete nucleotide sequences of segment A and B of each virus was determined as described before. Prior to challenge the isolates were propagated by one passage in RTG-2 cells.

In Vivo Challenge Experiment Atlantic Salmon Fry.

The challenge was conducted at VESO Vikan's research facility, Namsos, Norway. A total of 1020 Atlantic salmon (Salmo salar L.) fry of the AquaGen strain hatched at the VESO Vikan hatchery were included in the experiment. The fry had recently started to take feed (Micro 015, Ewos). At arrival at the research facilities, 20 fish were weighed and the average weight of the fry was 0.2 grams. The rest of the fish were divided into 4 tanks, each of 250 fry. After an acclimatization period of one week, the fry were starved one day prior to challenge. Fish were challenged by immersion with IPNV at a dose of 5×10⁴ TCID₅₀/ml in a total volume of 4 liters per tank. One tank was challenged with rNVI-15PA (Tank 1), one tank was challenged with rNVI-15PT (Tank 2), the third tank was challenged with a mix (50/50) of the two recombinant isolates (Tank 3), and one control tank was mock-infected by adding cell culture medium (Tank 4). The water was aerated during the challenge. After a period of 3 hours the water volume was reduced to 2 liters and normal flow was resumed. Mortality was recorded and dead fish were collected and frozen at −70° C. on daily basis. Sampling of ten fish from each tank was performed at ten days post challenge, and after this first sampling, ten fish were sampled from each tank once a month, up until six months post challenge.

Stress Exposures 6 Months Post Initial Challenge.

At six months post challenge, the remaining fish in each tank were divided into two parallels; A and B. Each of the parallels included 4 tanks with approximately 60 fish in each tank. Fish in tanks 1B-4B were subject to three stressful events during a period of one week. The stress was imposed by reducing water level to approximately ½ of the normal water level. In addition, the fish were chased for 15 minutes with a dipnet, at a moderate speed. Fish in parallels 1A-4A were not subject to any stress treatment (non-stressed).

After the stress treatment, samples were collected at the end of the stress period, then weekly for an additional 3 weeks post onset of stress (7 days between samplings). At each sampling, 12 fish from each group were sacrificed: six fish from each group were frozen at −70° C. and from six fish in each group the kidney was dissected and stored in RNAlater® (Qiagen).

Virus Re-Isolation from Persistently Infected Fish.

Fry samples stored at −70° C. without conservatives were added phosphate buffered saline (PBS) (1:5, weight/volume) and homogenized using a stomacher. 100 □l of this homogenate was transferred to 600 □l RLT buffer containing 2-mercaptoethanol (RNeasy Mini kit, Qiagen) and stored at −70° C. The rest of the homogenate was diluted 1:2 in L-15 medium supplemented with 2 mM L-glutamine and 50 □g ml⁻¹ gentamicin. After a brief centrifugation at 2500G for 10 minutes the supernatant were inoculated onto RTG-2 cells grown in 24 well plates in final dilutions of 1% and 0.1%, and incubated for one week at 15° C. The cell culture medium from the first passage was used to infect new monolayers.

The samples were considered negative when no CPE was observed after 1-week incubation of the second passage. RNA was isolated from the fish homogenate for all negative samples on cell culture using the RNeasy Mini kit (Qiagen) in accordance with the supplier's protocol, and RT-PCR was performed to amplify a 224-bp IPNV-specific DNA fragment, as described by Taksdal et al. (2001) with minor modifications. Qiagen's OneStep RT-PCR kit was used according to the manufacturer's instructions, with 0.5 μg RNA and 15 pmol each of primers IPNV-F and IPNV-R (Table 1) in a total reaction volume of 25 μl. The cycling conditions were 60° C. for 30 min., 95° C. for 15 min., followed by 45 cycles at 94° C. for 45 s, 57° C. for 45 s, 72° C. for 1 min., and finally 72° C. for 10 min. The PCR products were separated by agarose gel electrophoresis and visualized by staining with SYBR® Safe DNA gel stain (Invitrogen).

Three controls were included for each RT-PCR run: one positive and one negative tissue sample, and one negative control in which water was substituted for RNA.

TABLE 1

| | | | |
|---|---|---|---|
| Infectious | IPNV1F | Forward | SEQ ID No: 6 |
| pancreatic | | | ATCTGCGGAGTAGACATCAAAG |
| necrosis | IPNV2R | Reverce | SEQ ID No: 7 |
| virus, A | | | TGCAGTTCTTCGTCCATCCC |
| segment | | | |
| Infectious | A- | Forward | SEQ ID No: 8 |
| pancreatic | Sp500F | | GAGTCACAGTCCTGAATC |
| necrosis | | | |
| virus, A | | | |
| segment | | | |

Sequencing

RNA was isolated from homogenate stored on RLT buffer containing 2-mercaptoethanol (RNeasy Mini kit, Qiagen) stored at −70° C. after homogensation. RT-PCR was performed to amplify a IPNV-specific fragment (of VP2) using Qiagen's OneStep RT-PCR kit according to the manufacturer's instructions, with 0.5 μg RNA and 15 pmol each of primer A-Sp500F and A-Sp1689R (Table 1) in a total reaction volume of 25 μl. The cycling conditions were 50° C. for 30 min., 95° C. for 15 min., followed by 40 cycles at 94° C. for 45 s, 57° C. for 45 s, 72° C. for 2 min.15 s, and finally 72° C. for 10 min. The PCR products were separated by agarose gel electrophoresis and analyzed by staining with SYBR® Safe DNA gel stain.

To purify the DNA fragments from agarose gel, the Quantum Prep Freeze N' Squeeze DNA Gel Extraction Spin Column (BIO-RAD) was used according to the manufacturer's instructions. The recovered DNA was sequenced by a commercial sequencing service (Eurofins MWG operon) using primer A-Sp500F (Table 1). The sequence data were analyzed using VectorNTI software (Invitrogen). The chromatograms were examined as described previously (2) and briefly, by examining individual chromatograms obtained after sequencing (see below). The computer mouse was used to run over the chromatogram and thereby we obtained a relative level of the (different) nucleotides at the different positions. A threshold of 2% (of total nucleotides in each position) was set to allow exclusion of nucleotides present at very low levels.

Cloning and Nucleotide Sequencing.

Four samples with different mixtures of bases in position 217 and 221 were picked for cloning and sequencing. Taq polymerase-amplified PCR products from the sequencing step were cloned into a TOPO® Vector by using TOPO TA Cloning® according to the manufacturers' instructions (Invitrogen). The One Shot® Chemically Competent E. coli cells were transformed according to the protocol and 50 μl were spread on prewarmed agar plates containing 50 μg/ml ampicillin and pre-incubated for 30 min at 37° C. with 40 μl 40 mg/ml X-gal (Invitrogen) in dimethylformamide for white blue selection. 80-96 white colonies were picked from each Petri dish and placed one in each well on a 96 well agar plate with 150 mg apicillin (GATC Biotech). Plasmid were isolated and sequenced by a commercial sequencing company, GATC Biotech. The sequence data were analyzed using VectorNTI software.

### EXAMPLE 1

The nucleotide sequence of the VP2 gene is presented in SEQ ID NO: 1 and the amino acid sequence of the VP2 protein is represented by amino acid residue 1-442 of SEQ ID NO: 2.

Combination of plasmids pUC19NVI15VP2 and pUC19NVI15B resulted in the recovery of NVI-15PA (6). Ten times passage of NVI-15PA in CHSE-214 cell lines resulted in a mutation in position 221 in VP2 of NVI-15PA, A221T, and after plaque purification the rNVI-15PT isolate was recovered. The genomic RNAs of the recovered viruses were analyzed after RT-PCR amplification, and the sequence analysis of the RT-PCR products confirmed the expected mutations in the VP2 and VP1 regions of the chimeric and reassortant viruses. Furthermore, complete nucleotide sequences of segment A of both viruses were determined, and positions 252, 281, 282 and 319 had the following amino acids, VTNA, respectively (NVI-015PA is represented by SEQ ID No: 4).

### EXAMPLE 2

### Persistent Infection of Fry

The challenge dose used in this study was lower than what is used in standard challenge studies (5), since the purpose was to establish a persistent infection and to retain a high number of surviving fish. Of all the sampled fish during the experiment 94% were persistently infected. The cumulative mortality was 11% (PA), 11% (PA/PT) and 9.8% (PT) over the study period.

### EXAMPLE 3

### Re-Isolation and Characterization of IPNV from Infected Fry.

To document that the fish were actually infected and remained persistently infected we collected fry every 30 days from month 1-6 post infection. At each time point 10 fish were examined using cell culture and RT-PCR. Overall IPNV were reisolated by culture or detected by RT-PCR in 94% of the three groups (data not shown). All fish were positive by 6 months post challenge. Fish that were negative by culture were examined by RT-PCR. Progression from acute infection to persistence has been associated with increase in virus genome complexity. With the purpose to determine any complexity of the virus genomes of IPNV strains used here over the persistence period, RNA isolated from each of 5 fish from each group at all sampling points was amplified by RT-PCR and PCR-products were purified by gel electrophoresis and sent for sequencing.

### EXAMPLE 4

### Mutation Occurred Prior to Challenge In Vitro

The PA strain mutated in vitro (during initial culture) and appeared with a mutation in position 221; A221V (alanine exchanged with valine). This was not discovered prior to challenge and the “mixed” strain was thus used for challenge, meaning that the PA and PA/PT groups were challenged with a PA/PV and PA/PT/PV combination, respectively. The majority of mutations were found in residues 217, 221 and 247 (described earlier as hot spots for mutations in the VP2 of IPNV, Sp (5)). In addition positions 252, 281, 282 and 319 were examined for stability.

### EXAMPLE 5

### PA/PV Group

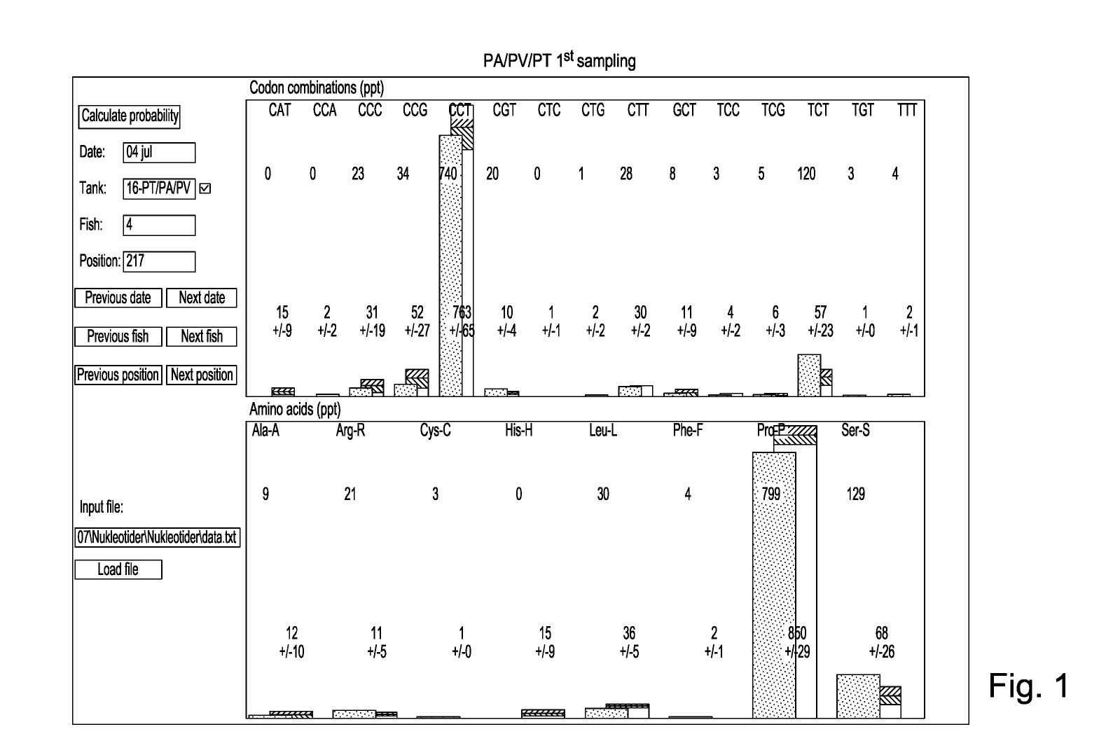

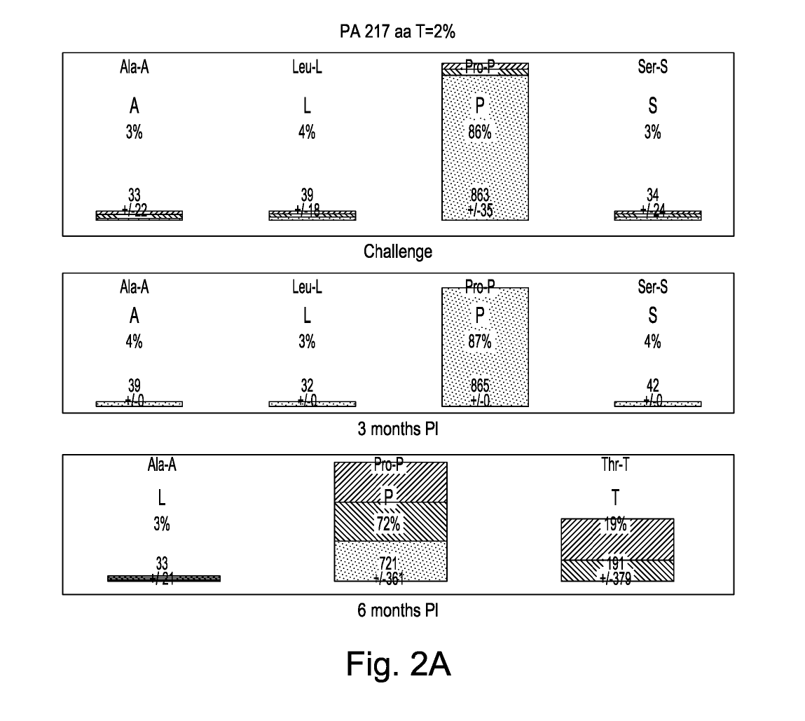

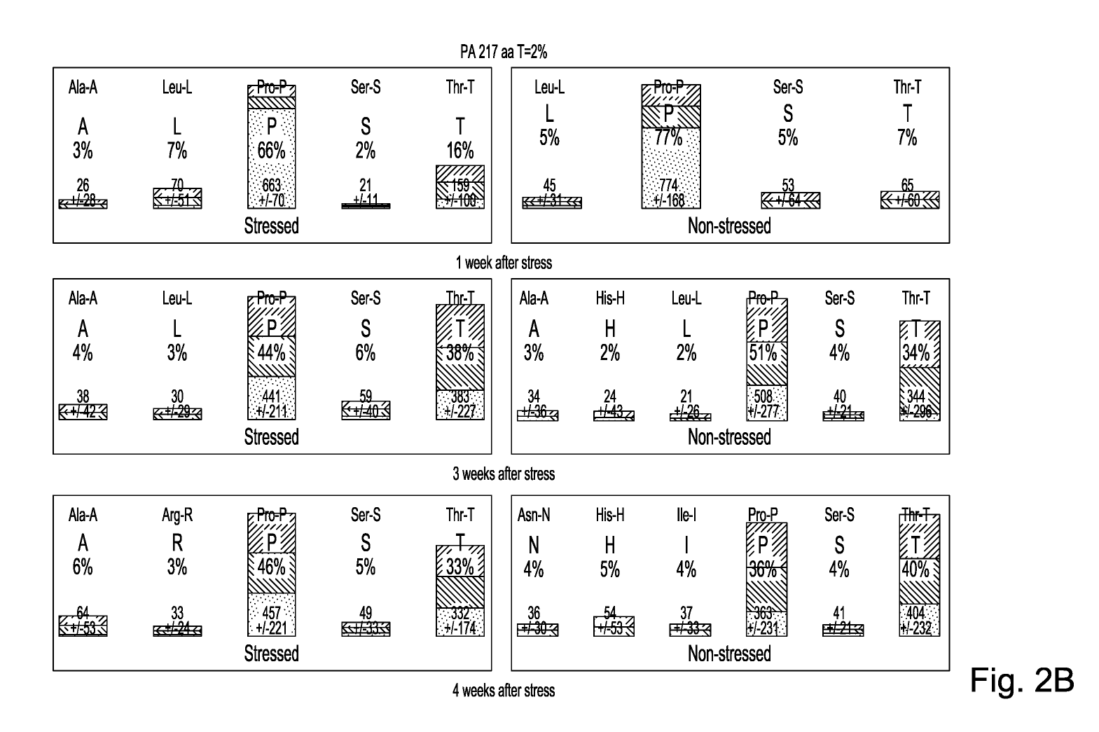

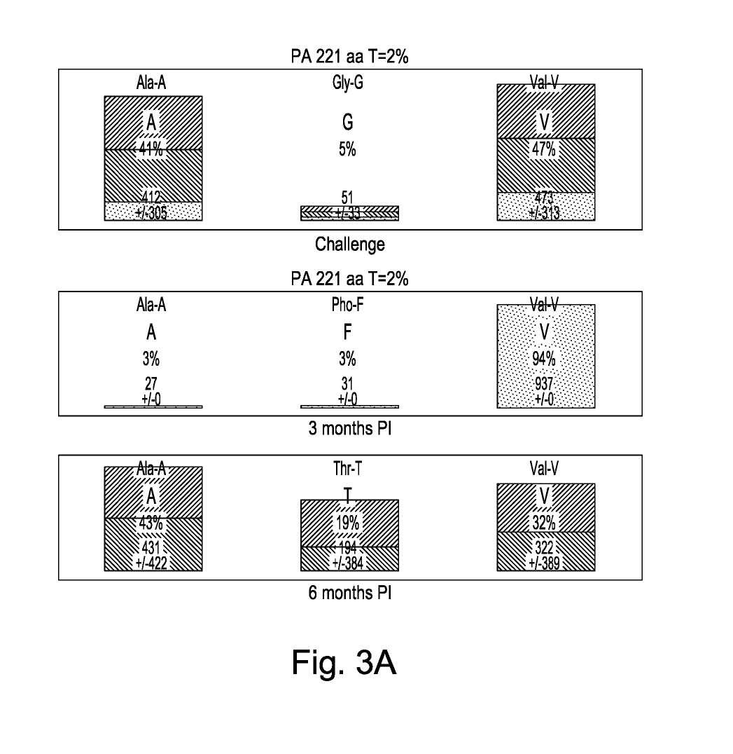

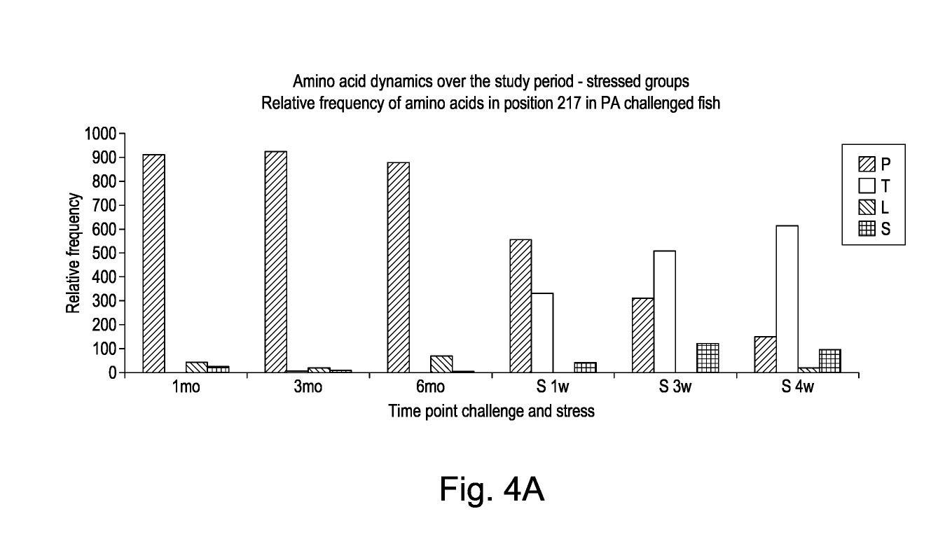

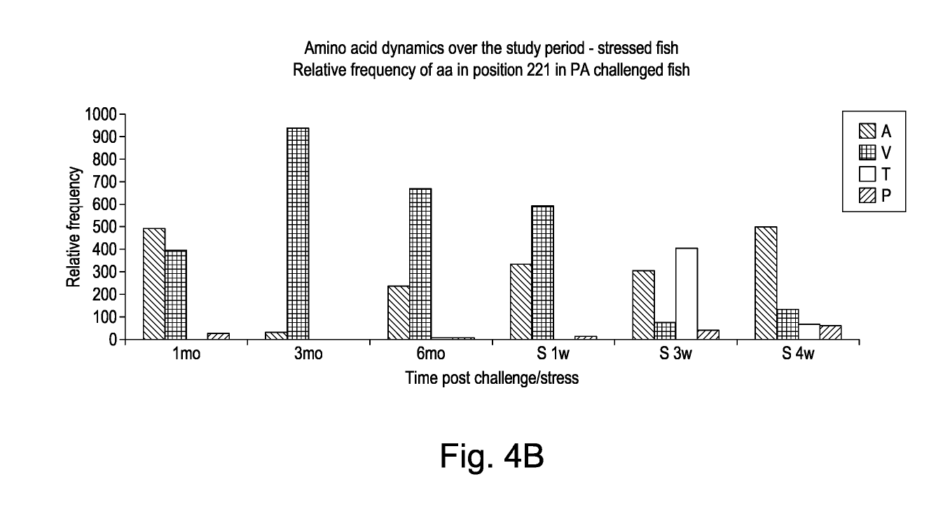

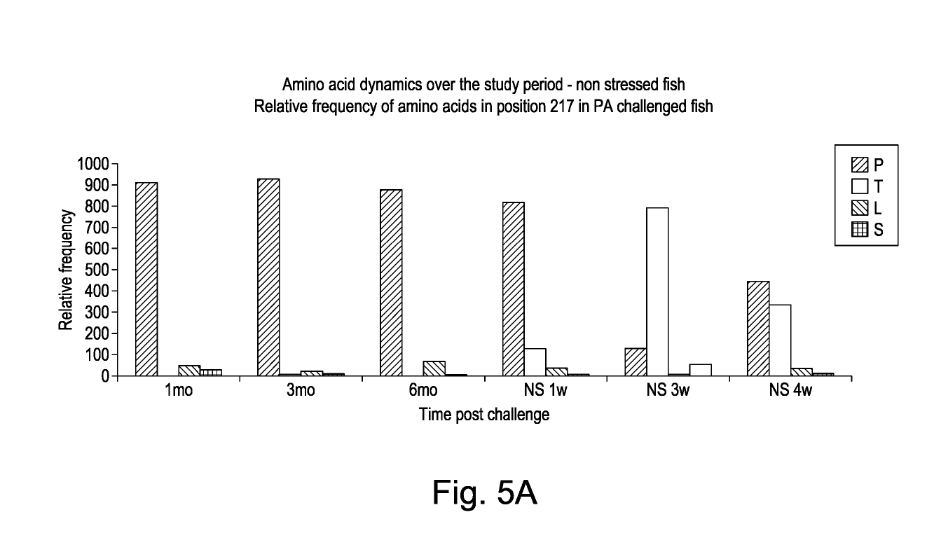

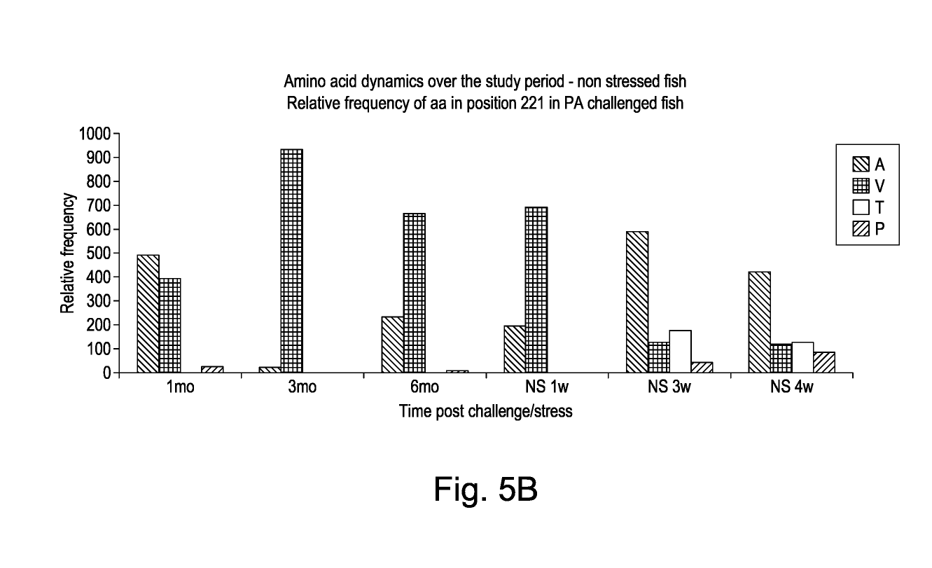

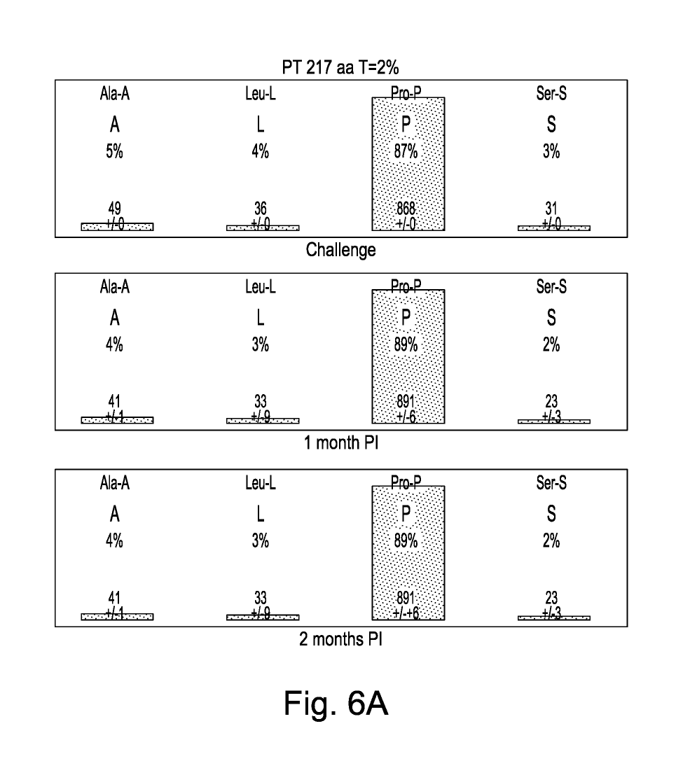

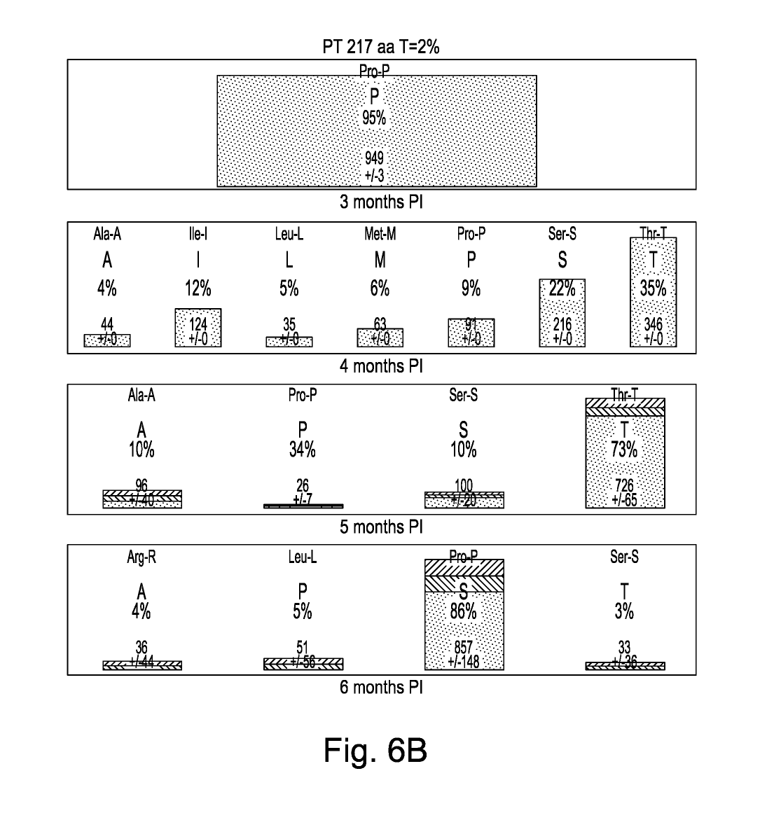

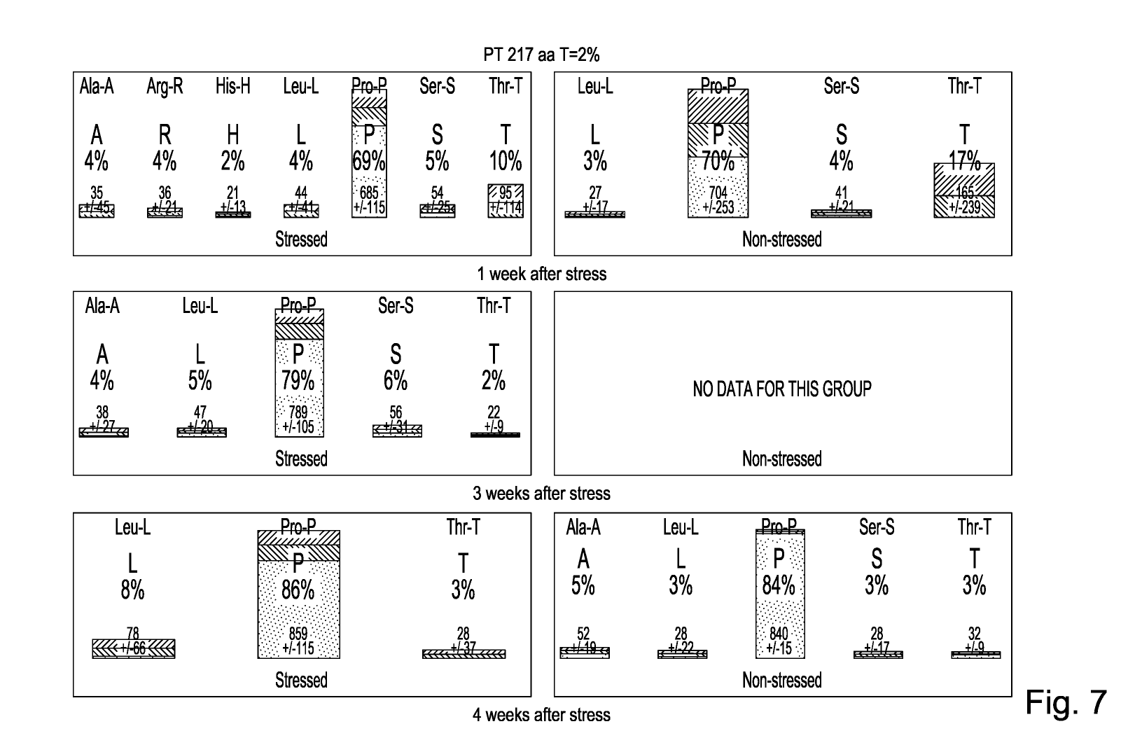

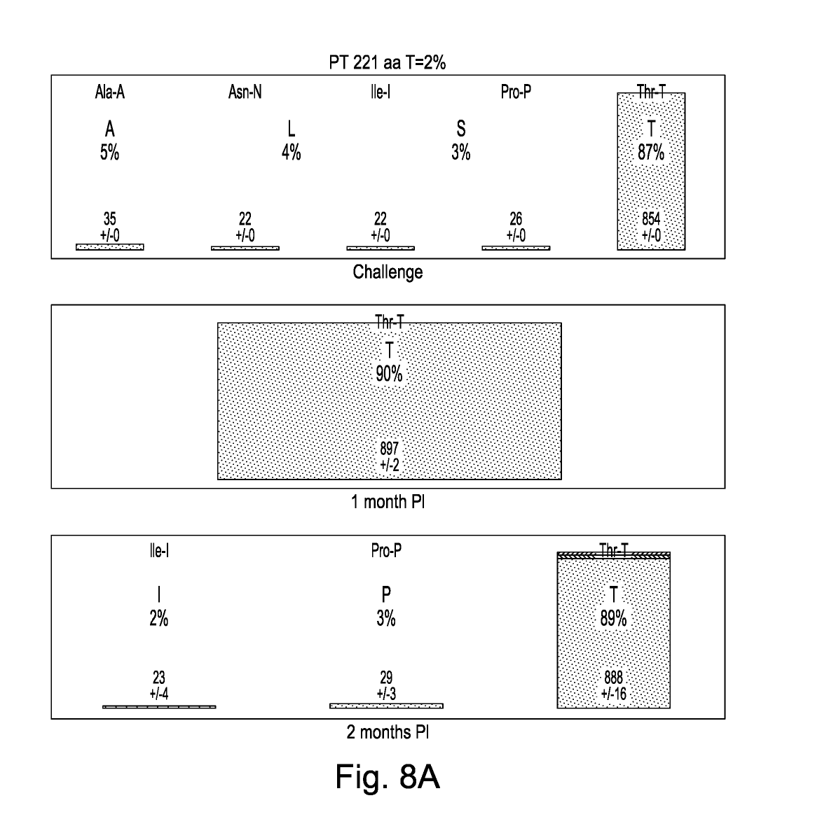

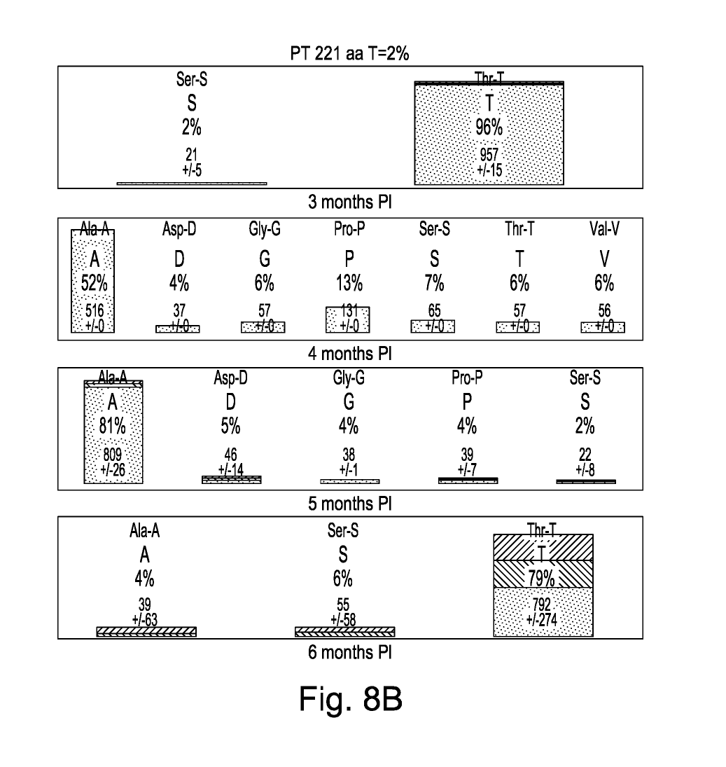

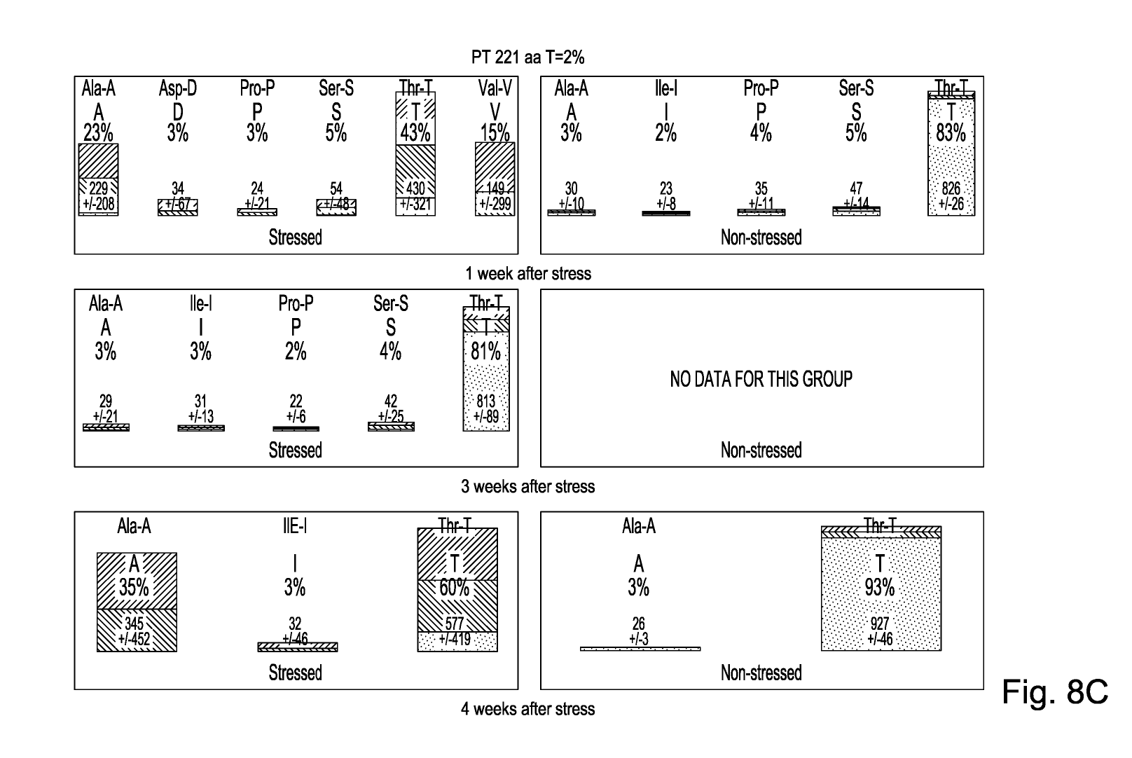

The variability in positions 217, 221 and 247 is described separately. The nucleotide/amino acid variation is based on examination of chromatograms after sequencing (as described above). The relative “amount” of nucleotides (and the corresponding) amino acids in each position was then entered into a “self-made” frequency calculation program that allowed comparison between the different strains and fish at the different positions, focusing on positions 217, 221 and 247 of VP2 (FIG. 1).

### EXAMPLE 6

### PA/PV/PT Dhallenged Fish

When fish infected with combinations of PA/PT (actually PA/PV/PT), a similar pictures is seen with increasing variability over time post challenge (not shown).

In addition to positions 217 and 221, there is also a marked variation in position 247 with variability increasing over time post challenge.

### Example 7

### Interaction Between Specific Codon Positions.

From the results presented above, non (PT)- or low-virulent (PA) virus strains used as single or combined source of infection of salmon fry result in increasing codon and amino acid variability over time post challenge, showing the instability of the virus genome during replication in a complex environment. Positions 217, 221 and 247 show particularly high variation with numerous combinations.

From the results presented it is difficult to observer or identify co-variation or correlation between mutations in the different codons. The inventors therefore examined interactions between nucleotide positions and also held the variation against mutations (synonymous and non-synonymous mutations) in other codons.

Codon 220 was found to show variation with links to the hypervariable codons 217, 221, and 247. A 3-level hierarchic statistical model was used to analyse the interactions between codons. There is a correlation between mutations in position 220.3 and mutations in certain positions in codons 217, 221, and 247. The specific interactions are depicted below and the following positions were found to correlate with position 220.3:

| Statistically significant: | | | | p-value | | |
|---|---|---|---|---|---|---|
| Position | .7349 | .0334622 | 21.96 | 0.000 | .6693152 | .8004847 |
| 247.1 \| | | | | | | |
| Position | −.3298807 | .042896 | −7.69 | 0.000 | −.4139553 | −.2458062 |
| 217.3 \| | | | | | | |
| Position | −.2560701 | .0389189 | −6.58 | 0.000 | −.3323497 | −.1797904 |
| 247.3 \| | | | | | | |
| Position | −.1231065 | .0219222 | −5.62 | 0.000 | −.1660732 | −.0801397 |
| 221.1 \| | | | | | | |
| Position | −.3353706 | .1399682 | −2.40 | 0.017 | −.6097031 | −.061038 |
| 217.2 \| | | | | | | |

| Borderline significance: | | | | p-value | | |
|---|---|---|---|---|---|---|
| Position | −.0650875 | .0355363 | −1.83 | 0.067 | −.1347375 | .0045624 |
| 217.1 \| | | | | | | |

| Not significant: | | | | p-value | | |
|---|---|---|---|---|---|---|
| Position | −.0024342 | .023253 | −0.10 | 0.917 | −.0480092 | .0431407 |
| 221.2 \| | | | | | | |
| Position | .0617472 | .1162098 | 0.53 | 0.595 | −.1660198 | .2895143 |
| 221.3 \| | | | | | | |
| Position | −.0020304 | .1273594 | −0.02 | 0.987 | −.2516501 | .2475894 |
| 247.2 \| | | | | | | |

The next step was to analyze if particular nucleotides in the indicated positions interact or dictates changes in other positions. We found that G and A in position 3 of codon 220 (220.3) give a highly significant statistical “interaction” value with 247.1 and 217.1, and these again impact on position 221.3. Functionally 220.3-G mutates to 220.3-A prior to changes in any of the other positions. These nucleotides (220.3, 247.1, 217.1 and 221.3) are “mixed” together in a very complicated pattern. The mathematic analysis gives the following;

Call: 1 m(formula=X220.3˜X217.1+X217.2+X217.3+X221.1+X221.2+X221.3+factor(Base)−1+Stress)

Residuals:

| Min | 1Q | Median | 3Q | Max |
|---|---|---|---|---|
| −0.85271 | −0.04887 | −0.01185 | 0.05626 | 0.88209 |

Coefficients:

| | Estimate | Std. Error | t value | Pr(>\|t\|) |
|---|---|---|---|---|
| X217.1 | 2.551e−01 | 4.070e−02 | 6.268 | 7.63e−10 *** |
| X217.2 | −6.807e−02 | 1.934e−01 | −0.352 | 0.725 |
| X217.3 | −3.780e−02 | 8.585e−02 | −0.440 | 0.660 |
| X221.1 | −4.039e−02 | 2.600e−02 | −1.553 | 0.121 |
| X221.2 | −4.253e−03 | 3.123e−02 | −0.136 | 0.892 |
| X221.3 | −1.276e−01 | 1.064e−01 | −1.199 | 0.231 |
| factor(Base) | A 1.131e−01 | 2.151e−02 | 5.257 | 2.14e−07 *** |
| factor(Base) | C −5.290e−03 | 1.799e−01 | −0.029 | 0.977 |
| factor(Base) | G 8.650e−01 | 2.243e−02 | 38.572 | <2e−16 *** |
| factor(Base) | T 5.023e−02 | 7.963e−02 | 0.631 | 0.528 |
| StressS | −1.399e−06 | 1.694e−02 | −8.26e−05 | 1.000 |

Signif. codes: 0 ‘***’ 0.001 ‘**’ 0.01 ‘*’ 0.05 ‘.’ 0.1 ‘ ’ 1

Further to this, the results show that stress is not a significant determinant for the interactions and mutations seen in these positions.

Residues 252, 281, 282 and 319 remained non-mutated through out the study with a preserved combination of VTNA as in the infecting virus strains (NVI-15PA is represented by SEQ ID No: 4 and NVI-025 is represented by SEQ ID No: 5).

### CONCLUSIONS

The findings here show that the IPN virus genome is highly unstable and readily mutates post infection in vivo. There are complex interactions between various codons and with positions 217, 221 and 247 being highly variable, where synonymous mutations in the 3^{rd} position of codon 220 impacts on the variability in the three defined codons. One interesting observation is that for both PA and PT infected fish, a combination of T₂₁₇A₂₂₁T₂₄₇ is found as a dominating virus clone.

### REFERENCES

1. Domingo, E., C. Escarmis, N. Sevilla, A. Moya, S. F. Elena, J. Quer, I. S. Novella, and J. J. Holland. 1996. Basic concepts in RNA virus evolution. FASEB journal : official publication of the Federation of American Societies for Experimental Biology 10:859-864.
2. Gadan, K., A. Sandtro, I. S. Marjara, N. Santi, H. M. Munang'andu, and O. Evensen. 2013. Stress-induced reversion to virulence of infectious pancreatic necrosis virus in naive fry of Atlantic salmon (Salmo salar L.). PloS one 8:e54656.
3. Lauring, A. S., and R. Andino. 2010. Quasispecies theory and the behavior of RNA viruses. Plos Pathogens 6:e1001005.
4. Santi, N., H. Song, V. N. Vakharia, and O. Evensen. 2005. Infectious pancreatic necrosis virus VP5 is dispensable for virulence and persistence. J Virol. 79:9206-9216.
5. Santi, N., V. N. Vakharia, and O. Evensen. 2004. Identification of putative motifs involved in the virulence of infectious pancreatic necrosis virus. Virology 322:31-40.
6. Song, H., N. Santi, O. Evensen, and V. N. Vakharia. 2005. Molecular determinants of infectious pancreatic necrosis virus virulence and cell culture adaptation. J Virol. 79:10289-10299.
7. Wright, S. 1931. Evolution in Mendelian Populations. Genetics 16:97-159.
8. Yao, K., and V. N. Vakharia. 1998. Generation of infectious pancreatic necrosis virus from cloned cDNA. J Virol. 72:8913-8920.
9. Molecular cloning: A laboratory manual: Edited by Sambrook, Fritsch and Maniatis—Fourth Edition: 2012

1. A live avirulent infectious pancreatic necrosis virus (IPNV) which does not revert to a virulent virus after at least 6 or at least 9 passages.

2. A live avirulent IPNV according to claim 1, wherein the amino acid in position 221 of the VP2 protein is Val and not Ala.

3. A live avirulent IPNV according to any one of claim 1 or 2, wherein position 3 of codon 220 is associated with a statistically significant interaction value with position 1 of nucleotide codons 247 (247.1), 217 (217.1) and position 3 of nucleotide codon 221 (221.3).

4. A live virulent IPNV according to claim 3, wherein the presence of G (guanine) or A (adenosine) at position 3 of codon 220 (codon 220.3) is associated with a statistically significant interaction value with position 1 of nucleotide codons 247 (247.1), 217 (217.1), and position 3 of nucleotide codon 221 (221.3).

5. A live avirulent IPNV according to claim 1 or claim 2, wherein the live avirulent IPNV incorporates a combination of codons selected from T₂₁₇A₂₂₁T₂₄₇.

6. (canceled)

7. (canceled)

8. A live avirulent IPNV according to claim 1 or claim 2, which when delivered by immersion at a titre of 5×10⁴TCID₅₀/ml to Atlantic salmon fry held in fresh water at a temperature of 12° C.
causes the fry to be virus positive measured by reisolation on RTG-2 cells;
causes the fry to be virus negative measured by immunohistochemistry;
provides the fry with protection against IPN disease as compared to non-infected fry; and
does not cause the fry to develop any signs of IPN disease.

9. A method for identifying or producing an avirulent IPNV, the method comprising the steps of:
a) characterising the distribution of nucleotides of VP2;
b) correlating step a) with the distribution of nucleotides at codons starting at codon 200 to codon 320; and
c) identifying or producing an avirulent IPNV.

10. A method for identifying or producing an avirulent IPNV according to claim 9, the method further comprising the steps of:
a) characterising the distribution of nucleotides of codons 220;
b) correlating step a) with the distribution of nucleotides at codons 247, 217 and 221; and
c) identifying or producing an avirulent IPNV.

11. A method for identifying or producing an avirulent IPNV according to claim 10, further comprising the steps of:
a) characterising position 3 of codon 220 (220.3);
b) correlating the findings from step a) with position 1 of codons 247 (247.1) and 217 (217.1) and position 3 of codon 221 (221.3) and
c) identifying or producing an avirulent IPNV.

12. (canceled)

13. A vaccine comprising the live avirulent IPNV according to claim 1 or claim 2.

14. (canceled)

15. The method according to claim 17, wherein the vaccine is administered to the subject by immersion, orally or by injection.

16. (canceled)

17. A method of treating or preventing the development of infectious pancreatic necrosis (IPN) disease comprising administering the vaccine of claim 13 to a subject in need of treatment or prevention thereof.


SEQUENCE LISTING (9 sequences)

SEQ ID NO: 1 - RNA, 1524 nt, Unknown
  Viruses
augaacacaaacaaggcaaccgcgaccuaccugaaauccauuaugcuuccagagacuggaccagcaagcaucccggacgacauaacggagagacacaucuuaaaacaagagaccucgucauacaaccuagaggucuccgaaucaggaaguggcauucuuguuuguuucccuggggcaccaggcucacgggucggugcacacuacagauggaaugcgaaccagacggggcuggaguucgaccaguggcuggagacgucgcaggaccugaagaaagccuucaauuacgggaggcugaucucaaggaaauacgacauccaaagcuccacacuaccggccggucucuacgcucugaacgggacgcucaacgcugccaccuucgagggcagucuguccgagguggagagccuggccuacaacagccugaugucccuaacaacgaacccccaggacaaagucaacaaccagcuggugacaaaaggagucacaguccugaaucuaccaacaggguucgacaaaccauacguccgccuagaggacgagacaccccagggucuccagucaaugaacggggccaagaugaggugcacagcugcaauugcaccgcggagguacgagaucgaccucccaucccaacgccuaccccccguuccugcgacaggaacccucaccacucucuacgagggaaacgccgacaucgucaacuccacaacagugacgggagacauaaacuucagucuggcagaacaacccgcaaacgagaccaaguucgacuuccagcuggacuucaugggccuugacaacgacgucccaguugucacaguggucagcuccgugcuggccucagaugacaacuacagaggagucucagccaagaugacccaguccaucccgaccgagaacaucacaaagccgaucaccagggucaagcugucauacaagaucaaccagcagacagaaaucggcaacgucgccacccugggcacaauggguccagcauccgucuccuucucaucagggaacggaaauguccccggcgugcucagaccaaucacacugguggccuaugagaagaugacaccgcuguccauccugaccguagcuggaguguccaacuacgagcugaucccaaacccagaacuccucaagaacauggugacacgcuauggcaaguacgaccccgaaggucucaacuaugccaagaugauccugucccacagggaagagcuggacaucaggacaguguggaggacagaggaguacaaggagaggaccagagucuucaacgagaucacggacuucuccagugaccugcccacgucaaaggcauggggcuggagagacauagucagaggaauucggaaagucgcagcuccuguacuguccacgcuguuuccaauggcagcaccacucauaggaauggcagaccaacucauuggagaucucacuaagaccaaugcagcaggcggaagguaccacuccauggccgcaggagggcgcuacaaagacgugcucgaguccugggca

SEQ ID NO: 2 - protein, 508 aa, Unknown
  Viruses
MNTNKATATYLKSIMLPETGPASIPDDTTERHILKQETSSYNLEVSESGSGILVCFPGAPGSRVGAHYRWNANQTGLEFDQWLETSQDLKKAFNYGRLISRKYDIQSSTLPAGLYALNGTLNAATFEGSLSEVESLAYNSLMSLTTNPQDKVNNQLVTKGVTVLNLPTGFDKPYVRLEDETPQGIQSMNGAKMRCTAAIAPRRYEIDLPSQRLPPVPATGTLTTLYEGNADIVNSTTVTGDINFSLAEQPANETKFDFQLDFMGLDNDVPVVTVVSSVLASDDNYRGVSAKMTQSIPTENITKPITRVKLSYKINQQTEIGNVATLGTMGPASVSFSSGNGNVPGVLRPITLVAYEKMTPLSILTVAGVSNYELIPNPELLKNMVTRYGKYDPEGLNYAKMILSHREELDIRTVWRTEEYKERTRVFNEITDFSSDLPTSKAWGWRDIVRGIRKVAAPVLSTLFPMAAPLIGMADQFIGDLTKTNAAGGRYHSMAAGGRYKDVLESWA

SEQ ID NO: 3 - DNA, 3097 bp, Unknown
  Viruses
ggaaagagagtttcaacgttagtggtaacccacgagcggagagctcttacggaggagctctccgtcgatggcgaaagccctttctaacaaacaaacaaacaatctatatcaatgcaagatgaacacaaacaaggcaaccgcaacttacctgaaatccattatgcttccagagactggaccagcaagcatcccggacgacataacggagagacacatcttaaaacaagagacctcgtcatacaacttagaggtctccgaatcaggaagtggcattcttgtttgtttccctggggcaccaggctcacggatcggtgcacactacagatggaatgcgaaccagacggggctggagttcgaccagtggctggagacgtcgcaggacctgaagaaagccttcaactacgggaggctgatctcaaggaaatatgacatccaaagctccacactaccggccggtctctatgctctgaacgggacgctcaacgctgccaccttcgaaggcagtctgtctgaggtggagagcctgacctacaacagcctgatgtccctaacaacgaacccccaggacaaagtcaacaaccagctggtgaccaaaggagtcacagtcctgaatctaccaacagggttcgacaaaccatacgtccgcctagaggacgagacaccccagggtctccagtcaatgaacggggccaagatgaggtgcacagctgcaattgcaccgcggaggtacgagatcgacctcccatcccaacgcctaccccccgttactgcgacaggagccctcaccactctctacgagggaaacgccgacatcgtcaactccacgacagtgacgggagacataaacttcagtctgacagaacaacccgcagtcgagaccaagttcgacttccagctggacttcatgggccttgacaacgacgtcccagttgtcacagtggtcagctccgtgctggccacaaatgacaactacagaggagtctcagccaagatgacccagtccatcccgaccgagaacatcacaaagccgatcaccagggtcaagctgtcatacaagatcaaccagcagacggcaatcggcaacgtcgccaccctgggcacaatgggtccagcatccgtctccttctcatcagggaacggaaatgtccccggcgtgctcagaccaatcacactggtggcctatgagaagatgacaccgctgtccatcctgaccgtagctggagtgtccaactacgagctgatcccaaacccagaactcctaaagaacatggtgacacgctatggcaagtacgaccccgaaggtctcaactatgccaagatgatcctgtcccacagggaagagctggacatcaggacagtgtggaggacagaggagtacaaggagaggaccagagtcttcaacgaaatcacggacttctccagtgacctgcccacgtcaaaggcatggggctggagagacatagtcagaggaattcggaaagtcgcagctcctgtactgtccacgctgtttccaatggcagcaccactcataggaatggcagaccaattcattggagatctcaccaagaccaacgcagcaggcggaaggtaccactccatggccgcaggagggcgctacaaagacgtgctcgagtcctgggcaagcggagggcccgacggaaaattctcccgagccctcaagaacaggctggagtccgccaactacgaggaagtcgagcttccacccccctcaaaaggagtcatcgtccctgtggtgcacacagtcaagagtgcaccaggcgaggcattcgggtccctggcaatcataattccaggggagtaccccgagcttctagatgccaaccagcaggtcctatcccacttcgcaaacgacaccgggagcgtgtggggcataggagaggacatacccttcgagggagacaacatgtgctacactgcactcccactcaaggagatcaaaagaaacgggaacatagtagtcgagaagatctttgctggaccaatcatgggtccctctgctcaactaggactgtccctactagtgaacgacatcgaggacggagttccaaggatggtattcaccggcgaaatcgccgatgacgaggagacaatcataccaatctgcggtgtagacatcaaagccatcgcagcccatgaacaagggctgccactcatcggcaaccaaccaggagtggacgaggaggtgcgaaacacatccctggccgcacacctgatccagaccggaaccctgcccgtacaacgcgcaaagggctccaacaagaggatcaagtacctgggagagctgatggcatcaaatgcatccgggatggacgaggaactgcaacgcctcctgaacgccacaatggcacgggccaaagaagtccaggacgccgagatctacaaacttcttaagctcatggcatggaccagaaagaacgacctcaccgaccacatgtacgagtggtcaaaagaggaccccgatgcactaaagttcggaaagctcatcagcacgccaccaaagcgccccgagaagcccaaaggaccagaccaacaccatgcccaagaggcgagagccacccgcatatcactggacgccgtgagagccggggcggacttcgccacaccggaatgggtcgcgctgaacaactaccgcggcccatctcccgggcagttcaagtactacctgatcactggacgagaaccagaaccaggcgacgagtacgaggactacataaaacaacccattgtgaaaccgaccgacatgaacaaaatcagacgtctagccaacagtgtgtacggcctcccacaccaggaaccagcaccagaggagttctacgatgcagttgcagctgtattcgcacagaacggaggcagaggtcccgaccaggaccaaatgcaagacctcagggagctcgcaagacagatgaaacgacgaccccggaacgccgatgcaccacggagaaccagagcgccagcggaaccggcaccgcccggacgctcaaggttcacccccagcggagacaacgctgaggtgtaacgactactctctttcctgactgatcccctggccaaaaccccggccccccagggggccccc

SEQ ID NO: 4 - protein, 508 aa, Unknown
  Viruses
MNTNKATATYLKSIMLPETGPASIPDDITERHILKQETSSYNLEVSESGSGILVCFPGAPGSRIGAHYRWNANQTGLEFDQWLETSQDLKKAFNYGRLISRKYDIQSSTLPAGLYALNGTLNAATFEGSLSEVESLTYNSLMSLTTNPQDKVNNQLVTKGVTVLNLPTGFDKPYVRLEDETPQGLQSMNGAKMRCTAAIAPRRYEIDLPSQRLPPVTATGALTTLYEGNADIVNSTTVTGDINFSLTEQPAVETKFDFQLDFMGLDNDVPVVTVVSSVLATNDNYRGVSAKMTQSIPTENITKPITRVKLSYKINQQTAIGNVATLGTMGPASVSFSSGNGNVPGVLRPITLVAYEKMTPLSILTVAGVSNYELIPNPELLKNMVTRYGKYDPEGLNYAKMILSHREELDIRTVWRTEEYKERTRVFNEITDFSSDLPTSKAWGWRDIVRGIRKVAAPVLSTLFPMAAPLIGMADQFIGDLTKTNAAGGRYHSMAAGGRYKDVLESWA

SEQ ID NO: 5 - protein, 508 aa, Unknown
  Viruses
MNTNKATATYLKSIMLPETGPASIPDDITERHILKQETSSYNLEVSESGSGILVCFPGAPGSRIGAHYRWNANQTGLEFDQWLETSQDLKKAFNYGRLISRKYDIQSSTLPAGLYALNGTLNAATFEGSLSEVESLTYNSLMSLTTNPQDKVNNQLVTKGVTVLNLPTGFDKPYVRLEDETPQGLQSMNGAKMRCTAAIAPRRYEIDLPSQRLPPVPATGALTTLYEGNADIVNSTTVTGDINFSLAEQPAVETKFDFQLDFMGLDNDVPVVTVVSSVLATNDNYRGVSAKMTQSIPTENITKPITRVKLSYKINQQTAIGNVATLGTMGPASVSFSSGNGNVPGVLRPITLVAYEKMTPLSILTVAGVSNYELIPNPELLKNMVTRYGKYDPEGLNYAKMILSHREELDIRTVWRTEEYKERTRVFNEITDFSSDLPTSKAWGWRDIVRGIRKVAAPVLSTLFPMAAPLIGMADQFIGDLTKTNAAGGRYHSMAAGGRHKDVLESWA

SEQ ID NO: 6 - DNA, 22 bp, Unknown
  Viruses
atctgcggagtagacatcaaag

SEQ ID NO: 7 - DNA, 20 bp, Unknown
  Viruses
tgcagttcttcgtccatccc

SEQ ID NO: 8 - DNA, 18 bp, Unknown
  Viruses
gagtcacagtcctgaatc

SEQ ID NO: 9 - DNA, 3097 bp, Unknown
  Viruses
ggaaagagagtttcaacgttagtggcaacccacgagcggagagctcctacggaggagctctccgtcgatggcgaaagccctttctaacaaacaaacaaacaatctatatcaatgcaagatgaacacaaacaaggcaaccgcaacttacctgaaatccattatgcttccagagactggaccagcaagcatcccggacgacataacggagagacacatcttaaaacaagagacctcgtcatacaacttagaggtctccgaatcaggaagtggcattcttgtttgtttccctggggcaccaggctcacggatcggtgcacactacagatggaatgcgaaccagacggggctggagttcgaccagtggctggagacgtcgcaggacctgaagaaagccttcaactacgggaggttgatctcaaggaaatacgacatccaaagctccacactaccggccggtctctatgctctgaacgggacgctcaacgctgccaccttcgagggcagtctgtctgaggtggagagcctgacctacaacagcctgatgtccctaacaacgaacccccaggacaaagtcaacaaccagctggtgaccaaaggagtcacagtcctgaatctaccaacagggttcgacaagccatacgtccgcctagaggacgagacaccccagggtctccagtcaatgaacggggccaagatgaggtgcacagctgcaattgcaccgcggaggtacgagatcgacctcccatcccaacgcctaccccccgttcctgcgacaggggccctcaccactctctacgagggaaacgccgacatcgtcaactccacgacagtgacgggagacataaacttcagtctggcagaacaacccgcagtcgagaccaagttcgacttccagctggacttcatgggccttgacaacgacgtcccagtcgtcacagtggtcagctccgtgctggccacaaatgacaactacagaggagtctcagccaagatgacccagtccatcccgaccgagaacatcacaaagccgatcaccagggtcaagctgtcatacaagatcaaccagcagacagcaatcggcaacgtcgccaccctgggcacaatgggtccagcatccgtctccttctcatcagggaacggaaatgtccccggcgtgctcagaccaatcacactggtggcctatgagaagatgacaccgctgtccatcctgaccgtagctggagtgtccaactacgagctgatcccaaacccagaactcctcaagaacatggtgacacgctatggcaagtacgaccccgaaggtctcaactatgccaagatgatcctgtcccacagggaagagctggacatcaggacagtgtggaggacagaggagtacaaggagaggaccagagtcttcaacgaaatcacggacttctccagtgacctgcccacgtcaaaggcatggggctggagagacatagtcagaggaattcggaaagtcgcagctcctgtactgtccacgctgtttccaatggcagcaccactcataggaatggcagaccaattcattggagatctcaccaagaccaacgcagcaggcggaaggtaccactccatggccgcaggagggcgccacaaagacgtgctcgagtcctgggcaagcggagggcccgacggaaaattctcccgagccctcaagaacaggctggagtccgccaactacgaggaagtcgagcttccacccccctcaaaaggagtcatcgtccctgtggtgcacacagtcaagagtgcaccaggcgaggcattcgggtccctggcaatcataattccaggggagtaccccgagcttctagatgccaaccagcaggtcctatcccacttcgcaaacgacaccgggagcgtgtggggcataggagaggacatacccttcgagggagacaacatgtgctacactgcactcccactcaaggagatcaaaagaaacgggaacatagtagtcgagaagatctttgctggaccaatcatgggtccctctgctcaactaggactgtccctacttgtgaacgacatcgaggacggagttccaaggatggtattcaccggcgaaatcgccgatgacgaggagacaatcataccaatctgcggtgtagacatcaaagccatcgcagcccatgaacaagggctgccactcatcggcaaccaaccaggagtggacgaggaggtgcgaaacacatccctggccgcacacctgatccagaccggaaccctgcccgtacaacgcgcaaagggctccaacaagaggatcaagtacctgggagagctgatggcatcaaatgcatccgggatggacgaggaactgcaacgcctcctgaacgccacaatggcacgggccaaagaagtccaggacgccgagatctacaaacttcttaagctcatggcatggaccagaaagaacgacctcaccgaccacatgtacgagtggtcaaaagaggaccccgatgcactaaagttcggaaagctcatcagcacgccaccaaagcaccctgagaagcccaaaggaccagaccaacaccacgcccaagaggcgagagccacccgcatatcactggacgccgtgagagccggggcggacttcgccacaccggaatgggtcgcgctgaacaactaccgcggcccatctcccgggcagttcaagtactacctgatcactggacgagaaccagaaccaggcgacgagtacgaggactacataaaacaacccattgtgaaaccaaccgacatgaacaaaatcagacgtctagccaacagtgtgtacggcctcccacaccaggaaccagcaccagaggagttctacgatgcagttgcagctgtattcgcacagaacggaggcagaggtcccgaccaggaccaaatgcaagacctcagggagctcgcaagacagatgaaacgacgaccccggaacgccgatgcaccacggagaaccagagcgccagcggaaccggcaccgcccggacgctcaaggttcacccccagcggagacaacgctgaggtgtaacgactactctctttcctgactgatcccctggccaaaaccccggccccccagggggccccc